(19) United States
(12) Patent Application Publication
(10) Pub. No.: US 2009/0188008 A1
(43) Pub. Date: Jul. 23, 2009

(54) COMPOSITIONS AND METHODS FOR THE SUPPRESSION OF TARGET POLYNUCLEOTIDES

(75) Inventor: Michael Lassner, Urbandale, IA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignee: Pioneer Hi-Bred International, Inc., Johnston, IA (US)

(21) Appl. No.: 12/351,093

(22) Filed: Jan. 9, 2009

Related U.S. Application Data
(60) Provisional application No. 61/021,676, filed on Jan. 17, 2008.

(51) Int. Cl. C12N 15/82; C12N 5/04; A01H 5/00

(52) U.S. Cl. 800/279; 435/419; 435/415; 435/416; 435/412; 800/302

(57) ABSTRACT

Methods and compositions which increase the concentration of an inhibitory RNA specific for a target sequence in a cell are provided. In one embodiment, the methods and compositions employ a first polynucleotide comprising a silencing element for a target pest sequence operably linked to a promoter active in the plant cell; and, a second polynucleotide comprising a suppressor enhancer element comprising the target pest sequence or an active fragment or variant thereof operably linked to a promoter active in the plant cell. The combined expression of the silencing element with the target pest sequence, or an active variant or fragment thereof, leads to the amplification of the inhibitory RNA produced from the silencing element over the achievable with only the expression of the silencing element alone. Thus, the various methods and compositions of the invention provide improved methods for the delivery of inhibitory RNA to a target organism.

### CROSS-REFERENCE TO RELATED APPLICATIONS

This application claims priority to U.S. Provisional Application No. 61/021,676; filed Jan. 17, 2008 which is herein incorporated by reference in their entirety.

### FIELD OF THE INVENTION

The present invention relates generally to methods of molecular biology and gene silencing.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 366608seqlist.txt, a creation date of Jan. 5, 2009, and a size of 88 Kb. The sequence listing filed via EFS-Web is part of the specification and is hereby incorporated in its entirety by reference herein.

### BACKGROUND OF THE INVENTION

RNA interference (RNAi) is a method for selectively disrupting gene function in a targeted organism. The utility of this method for genetic dissection of cellular processes in vitro is well established. Application of RNAi to insect whole organisms has been largely limited to the fruit fly Drosophila melanogaster via P element mediated germline transformation (along with a limited number of reports of RNAi experiments in other insects based on injection of dsRNA into the insect hemocoel). Methods are needed in the art to enhance targeted suppression of the sequences of interest in various organisms.

Insect pests are a serious problem in agriculture. They destroy millions of acres of staple crops such as corn, soybeans, peas, and cotton. Yearly, these pests cause over $100 billion dollars in crop damage in the U.S. alone. In an ongoing seasonal battle, farmers must apply billions of gallons of synthetic pesticides to combat these pests. Other methods employed in the past delivered insecticidal activity by microorganisms or genes derived from microorganisms expressed in transgenic plants. For example, certain species of microorganisms of the genus Bacillus are known to possess pesticidal activity against a broad range of insect pests including Lepidoptera, Diptera, Coleoplera, Hemiptera, and others. In fact, microbial pesticides, particularly those obtained from Bacillus strains, have played an important role in agriculture as alternatives to chemical pest control. Agricultural scientists have developed crop plants with enhanced insect resistance by genetically engineering crop plants to produce insecticidal proteins from Bacillus. For example, corn and cotton plants genetically engineered to produce Cry toxins (see, e.g., Aronson (2002) Cell Mol. Life. Sci. 59(3):417-425; Schnepf et al. (1998) Microbiol. Mol. Biol. Rev. 62(3):775-806) are now widely used in American agriculture and have provided the farmer with an alternative to traditional insect-control methods. However, these Bt insecticidal proteins only protect plants from a relatively narrow range of pests. Moreover, these modes of insecticidal activity provided varying levels of specificity and, in some cases, caused significant environmental consequences. Thus, there is an immediate need for alternative methods to control pests.

### BRIEF SUMMARY OF THE INVENTION

Methods and compositions which increase the concentration of an inhibitory RNA specific for a target sequence in a cell are provided. In one embodiment, the methods and compositions employ a first polynucleotide comprising a silencing element for a target pest sequence operably linked to a promoter active in the plant cell; and, a second polynucleotide comprising a suppressor enhancer element comprising the target pest sequence or an active fragment or variant thereof operably linked to a promoter active in the plant cell. The combined expression of the silencing element with the suppressor enhancer element leads to the increased amplification of the inhibitory RNA produced from the silencing element over that achievable with the expression of the silencing element alone.

Further provided is a method for controlling a pest. The method comprises feeding to the pest a plant cell comprising a first polynucleotide comprising a silencing element for a pest target sequence and a second polynucleotide comprising the suppressor enhancer element.

Compositions comprising plants, plant cells, plant parts, seeds and vectors comprising a first polynucleotide comprising a silencing element for a target pest sequence operably linked to a promoter active in the plant cell; and, a second polynucleotide comprising a suppressor enhancer element comprising a target pest sequence or an active variant or fragment thereof operably linked to a promoter active in the plant cell are further provided.

### BRIEF DESCRIPTION OF DRAWINGS

FIG. 1 provides a non-limiting illustration of a suppression construct.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Methods and compositions which increase the concentration of an inhibitory RNA (RNAi) specific for a target sequence in a cell are provided. In one embodiment, the methods and compositions employ a first polynucleotide comprising a silencing element for a target pest sequence operably linked to a promoter active in the plant cell; and, a second polynucleotide comprising a suppressor enhancer comprising the target pest sequence or an active variant or fragment thereof operably linked to a promoter active in the plant cell. The combined expression of the silencing element with suppressor enhancer element leads to an increased amplification of the inhibitory RNA produced from the silencing element over that achievable with only the expression of the silencing element alone. In addition to the increased amplification of the specific RNAi species itself, the methods and compositions further allow for the production of a diverse population of RNAi species that can enhance the effectiveness of disrupting target gene expression. As such, when the suppressor enhancer element is expressed in a plant cell in combination with the silencing element, the methods and composition can allow for the systemic production of RNAi throughout the plant; the production of greater amounts of RNAi than would be observed with just the silencing element construct alone; and, the improved loading of RNAi into the phloem of the plant, thus providing better control of phloem feeding insects by an RNAi approach. Thus, the various methods and compositions of the invention provide improved methods for the delivery of inhibitory RNA to the target organism.

As used herein, by “controlling a pest” or “controls a pest” is intended any affect on a pest that results in limiting the damage that the pest causes. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack, or deterring the pests from eating the plant.

By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen is minimized or lessened.

Reducing the level of expression of the target polynucleotide or the polypeptide encoded thereby in the pest results in the suppression, control, and/or killing the invading pathogenic organism. Reducing the level of expression of the target sequence of the pest will reduce the disease symptoms resulting from pathogen challenge by at least about 2% to at least about 6%, at least about 5% to about 50%, at least about 10% to about 60%, at least about 30% to about 70%, at least about 40% to about 80%, or at least about 50% to about 90% or greater. Hence, the methods of the invention can be utilized to protect plants from disease.

Assays that measure the control of a pest are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. See, for example, Thomma et al. (1998) Plant Biology 95:15107-15111, herein incorporated by reference. See, also the examples below.

In one embodiment, compositions and methods for protecting plants from a plant pest are provided. In specific embodiments, the RNAi produced in the methods does not reduce the level of expression of a plant sequence or other sequences from a non-targeted animal, including, but not limited to, predators of the pests (i.e., ladybird larvae, anthocorid bugs, lacewing, parasitic wasps, or hoverfly larvae) or animals such as humans, mammals, birds, amphibians, reptiles, etc.

Pathogens (pests) of the invention include, but are not limited to, viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Fungal pathogens, include but are not limited to, Colletotrichum graminocola, Diplodia maydis, Fusarium graminearum, and Fusarium verticillioides. Specific pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassicicola, Pythium ultimum, Peronospora parasitica, Fusarium roseum, Alternaria alternata; Alfalfa: Clavibacter michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Verticillium albo-atrum, Xanthomonas campestris p.v. alfalfae, Aphanomyces euteiches, Stemphylium herbarum, Stemphylium alfalfae, Colletotrichum trifolii, Leptosphaerulina briosiana, Uromyces striatus, Sclerotinia trifoliorum, Stagonospora meliloti, Stemphylium botryosum, Leptotrichila medicaginis; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Plasmopora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Colletotrichum graminicola, Fusarium moniliforme var. subglutinans, Erwinia stewartii, F. verticillioides, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Scierophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, C. sublineolum, Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalputs lignosellus, lesser cornstalk borer; Diatraea saccharalis, sugarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize bilibug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrolica undecimpunclata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctala, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogranima exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, red legged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

In one embodiment, the pest is from the Hemiptera order. The Hemiptera order comprises four suborders: the Sternorrhyncha (e.g. aphids, whiteflies), Auchenorrhyncha (e.g. cicadas, leafhoppers), Coleorrhyncha, and Heteroptera (e.g. true bugs). Accordingly, the compositions and methods are useful in protecting plants against any member of the Hemiptera order including those of the family Cicadellidae, Membracidae, Fulgoridae, Coccidae, Aphididae, Lygaeidae, Pentatomidae, and Miridae.

In other embodiments, the pest is from the Lygus genus. The Lygus genus comprises over 40 species of plant feeding insects in the family Miridae. As used herein, the term “Lygus” or “Lygus Bug” is used to refer to any member of the Lygus genus. Accordingly, the compositions and methods are also useful in protecting plants against any Lygus including, for example, Lygus adspersus, Lygus alashanensis, Lygus borealis, Lygus elisus, Lygus gemellatus, Lygus Hesperus, Lygus lineolaris, or Lygus rugulipennis. In particular embodiment, methods control Lygus Hesperus.

In other embodiments, the pest is from the Lepidoptera order. Caterpillars and related forms of lepidopteran insects comprise an important group of plant-feeding agricultural pests, especially during the larvae stage of growth. Feeding methods of Lepidoptera larvae typically include chewing plants or plant parts. As used herein, the term “Lepidoptera” is used to refer to any member of the Lepidoptera order. In particular embodiments, compositions and methods of the invention control Lepidoptera larvae (i.e. caterpillars). Accordingly, the compositions and methods are also useful in protecting plants against any Lepidoptera including, for example, Pieris rapae, Pectinophora gossypiella, Synanthedon exitiosa, Melittia cucurbitae, Cydia pomonella, Grapholita molesta, Ostrinia nubilalis, Plodia interpunctella, Galleria niellonella, Manduca sexta, Manduca quinquemaculata, Lymantria dispar, Euproctis chrysorrhoea, Trichoplusia ni, Mamestra brassicae, Agrolis ipsilon, or Spodoplera littoralis. In particular embodiments, the pest is Spodoptera frugiperda.

In other embodiments, the pest is from Aphidoidea. As used herein, the term “Aphididae” or “Aphid” is used to refer to any member of the Aphididae family. Accordingly, the compositions and methods are also useful in protecting plants against any Aphididae including, for example, peach-potato aphid Myzus persicae, the bean aphid Aphis fabae, the pea aphid Acyrthosiphum pisun, the cabbage aphid Brevicoryne brassicae, the grain aphid Sitobion avenae, the rose-grain aphid Metopolophium dirhodum, the Russian wheat aphid Diuraphis noxia (Mordvilko), the English grain aphid Macrosiphum avenae, the greenbug aphid Schizaphis graminum (Rondani), the carrot aphid Cavariella aegopodii, the potato aphid Macrosiphum euphorbiae, the groundnut aphid Aphis craccivora, the cotton aphid Aphis gossypii, the black citrus aphid Toxoptera aurantii, the brown citrus apid Toxoptera ciidius, the willow aphid Cavariella spp., the corn leaf aphid Rhopalosiphuni maidis, the aphid Rhopalosiphum padi, the willow leaf aphids Chaitophorus spp., the black pine aphids Cinara spp., the sycamore aphid Drepanosiphum platanoides, the spruce aphids Elatobium spp., Aphis citricola, Lipaphis pserudobrassicae (turnip aphid), Nippolachnus piri, the foxglove aphid Aulacorthum solani, the asparagus aphid Brachycorynella asparagi, the brown ambrosia aphid Uroleucon ambrosiae, the buckthorn aphid Aphis nasturtii, the corn root aphid Aphis maidiradicis, the cresentmarked lily aphid Neomyzus circumflexes, the goldenglow aphid Dactynolus rudbeckiae, the honeysuckle and parsnip aphid Hyadaphis foeniculi, the leafcurl plum aphid Brachycaudus helichrysi, the lettuce root aphid Pemphigus bursarius, the mint aphid Ovatus crataegarius, the artichoke aphid Capitophorus elaeagni, the onion aphid Neotoxoptera formosana, the pea aphid Macrosiphum pisi, the rusty plum aphid Hysteroneura setariae, the shallot aphid Myzus ascalonicus, the solanum root aphid Smynthurodes betae, the sugarbeet root aphid Pemphigus betae, the tulip bulb aphid Dysaphis fulipae, the western aster root aphid Aphis armoraciae, the white aster root aphid Prociphilus erigeronensis. In particular embodiments, methods control the soybean aphid Aphis glycines.

In one embodiment, the pest is a plant sap-sucking insect. As used herein, “plant sap-sucking insects” are insects which feed on plants using their sharp mouth parts which can be inserted into a plant to take fluid from the plant vascular system. In one embodiment, these are insects feeding directly on the fluids in the plant vascular system. In the insertion site, plant cells can also be damaged which may or may not be used as a food source by the plant sap-sucking insect. These insects are plant pests because their feeding reduces the vitality of the crop they feed on and they can transmit viral disease. Also, such sap-sucking insects can create a sugar-rich fluid named honeydew that accumulates on lower plant parts and such parts soon become covered by certain black or brown fungi known as sooty molds, hence interfering with photosynthesis.

Included in such plant sap-sucking insects are aphids or Homopteran insects of the Aphididae, and plant sap-sucking insects as used herein include but are not limited to the peach-potato aphid Myzus persicae, the bean aphid Aphis fabae, the pea aphid Acyrthosiphumpisun, the cabbage aphid Brevicoryne brassicae, the grain aphid Sitobion avenae, the rose-grain aphid Metopolophium dirhodum, the Russian wheat aphid Diuraphis noxia (Mordvilko), the English grain aphid Macrosiphum avenae, the greenbug aphid Schizaphis graminum (Rondani), the carrot aphid Cavariella aegopodii, the potato aphid Macrosiphum euphorbiae, the groundnut aphid Aphiscraccivora, the cotton aphid Aphis gossypii, the black citrus aphid Toxoptera aurantii, the brown citrus apid Toxoptera ciidius, the willow aphid Cavariella spp., the corn leaf aphid Rhopalosiphum maidis, the aphid Rhopalosiphum padi, the willow leaf aphids Chaitophorus spp., the black pine aphids Cinara spp., the Sycamore Aphid Drepanosiphum platanoides, the Spruce aphids Elatobium spp., Aphis citricola, Lipaphis as Laodelphax striatellus (small brown planthopper), Nilaparvata lugens (rice brown plant hopper) and Sogatella furcifera (white-backed rice planthopper), and Deltocephalidae (or leafhoppers) such as Flexamia DeLong spp., Nepholettix cincticeps and Nephotettix virescens, Amrasca bigutulla, and the potato leafhopper Empoasca filament. Also included are scales (also named scale insects) such as Aonidiella aurantii (California red scale), Comstockaspis perniciosa (San Jose scale), Unaspis citri (citrus snow scale), Pseudaulacaspis pentagona (white peach scale), Saissetia oleae (brown olive scale), Lepidosaphes beckii (purple scale), Ceroplastes rubens (red wax scale) and Icerya purchasi (cottonycushion scale), besides Tingidae (or lace bugs) and Psyllidae insects, and spittle bugs.

Further included as plant sap-sucking insects are Heteropteran insects and Hemipteran insects of the Auchenorrhyncha that feed from the plants' vascular system, such as sap-sucking insects of the Cicadoidea (such as Cicadas), Cercopoidea (spittlebugs or froghoppers), Membracoidea (leafhoppers and treehoppers), and Fulgoroidea (planthoppers), e.g., the cotton seed sucker bug Dysdercus peruvianus (Heteroptera, Pyrrhocoridae), the apple dimpling bug, Campylomma liebknechti (Hemiptera: Miridae) and the greenmirid, Creontiades dilutus which are cotton sucking insect pests, and the Lygusbugs (Hemiptera: Miridae, e.g., Lygus hesperus).

### II. Target Sequences

As used herein, a “target sequence” comprises any sequence in the pest that one desires to decrease the level of expression. In specific embodiments, the target sequence is from a pest. In further embodiments, decreasing the level of the target sequence in the pest controls the pest. For instance, the target sequence can be essential for growth and development. While the target sequence can be expressed in any tissue of the pest, in specific embodiments of the invention, the sequences targeted for suppression in the pest are expressed in cells of the gut tissue of the pest, cells in the midgut of the pest, and cells lining the gut lumen or the midgut. Such target sequences can be involved, for example, in gut cell metabolism, growth or differentiation.

Non-limiting examples of target sequences of the invention include polynucleotides as disclosed in U.S. Provisional Application 61/021,685, entitled “Compositions and Methods for the Suppression of Target Polynucleotides from Lygus”, filed Jan. 17, 2008, U.S. Provisional Application 61/021,699, entitled “Compositions and Methods for the Suppression of Target Polynucleotides from Lepidoptera”, filed Jan. 17, 2008; and U.S. Provisional Application 61/108,924, entitled “Compositions and Methods for the Suppression of Target Polynucleotides from the Family Aphididea, filed Oct. 28, 2008. Each of these applications is herein incorporated by reference in their entirety. Additional target pest sequences which can be targeted employing the methods and compositions of the invention are further disclosed in, for example, WO 2005/049841, US 2005/0095199, WO 01/37654, and WO 2005/110068, each of which is herein incorporated by reference in their entirety. Additional target sequences are set forth in SEQ ID NOS:1-58.

### III. Polynucleotides Comprising Suppressor Enhancer Elements

In the methods of the invention, the combined expression of a silencing element and a suppressor enhancer element comprising the targeted sequence, or an active fragment or variant thereof, leads to an increased amplification of the inhibitory RNA produced from the silencing element over that achievable with only the expression of the silencing element alone. As used herein, a “suppressor enhancer element” comprises a polynucleotide comprising the target sequence to be suppressed or an active fragment or variant thereof.

It is recognize that the suppressor enhancer element need not be identical to the target sequence, but rather, the suppressor enhancer element can comprise a variant of the target sequence, so long as the suppressor enhancer element has sufficient sequence identity to the target sequence to allow for an increased level of the RNAi produced by the silencing element over that achievable with only the expression of the silencing element. Similarly, the suppressor enhancer element can comprise a fragment of the target sequence, wherein the fragment is of sufficient length to allow for an increased level of the RNAi produced by the silencing element over that achievable with only the expression of the silencing element.

Further multiple suppressor enhancer elements for the same target sequence can be employed. For example, the suppressor enhancer elements employed can comprise fragments of the target sequence derived from different region of the target sequence (i.e., from the 3'UTR, coding sequence, intron, and/or 5'UTR).

### IV. Silencing Elements

By “silencing element” is intended a polynucleotide which is capable of reducing or eliminating the level or expression of a target polynucleotide or the polypeptide encoded thereby. In specific embodiments, the silencing element, when ingested by a pest, specifically reduces or eliminates the level of a target pest sequence. The silencing element employed can reduce or eliminate the expression level of the target sequence by influencing the level of the target RNA transcript or, alternatively, by influencing translation and thereby affecting the level of the encoded polypeptide. Methods to assay for functional silencing elements that are capable of reducing or eliminating the level of a sequence of interest are disclosed elsewhere herein. A single polynucleotide employed in the methods of the invention can comprises one or more silencing elements to the same or different target polynucleotide. As used herein, an “inhibitory RNA” or “RNAi” is intended an RNA molecule which is capable of reducing or eliminating the level of expression of a target polynucleotide or polypeptide encoded thereby in a sequence specific manner.

In specific embodiments, the target sequence is not a plant endogenous gene. In other embodiments, while the silencing element controls pests, preferably the silencing element has no effect on the normal plant or plant part.

As discussed in further detail below, silencing elements can include, but are not limited to, a double stranded RNA, a miRNA, or a hairpin suppression element. Non-limiting examples of silencing elements that can be employed in the methods and compositions of the invention include polynucleotides as disclosed in U.S. Provisional Application 61/021,685, entitled “Compositions and Methods for the Suppression of Target Polynucleotides from Lygus”, filed Jan. 17, 2008; U.S. Provisional Application 61/021,699, entitled “Compositions and Methods for the Suppression of Target Polynucleotides from Lepidoptera”, filed Jan. 17, 2008; and U.S. Provisional Application 61/108,924, entitled “Compositions and Methods for the Suppression of Target Polynucleotides from the Family Aphididea”, filed Oct. 28, 2008. Each of these applications is herein incorporated by reference in their entirety. Additional sequence pest sequences which can be targeted employing the methods and compositions of the invention are further disclosed in, for example, WO 2005/049841, US 2005/0095199, WO 01/37654, and WO 2005/110068, each of which is herein incorporated by reference in their entirety.

By “reduces” or “reducing” the expression level of a polynucleotide or a polypeptide encoded thereby is intended to mean, the polynucleotide or polypeptide level of the target sequence is statistically lower than the polynucleotide level or polypeptide level of the same target sequence in an appropriate control which is not exposed to the silencing element and the suppressor enhancer element. In particular embodiments of the invention, reducing the polynucleotide level and/or the polypeptide level of the target sequence in a pest according to the invention results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control pest. Methods to assay for the level of the RNA transcript, the level of the encoded polypeptide, or the activity of the polynucleotide or polypeptide are discussed elsewhere herein.

In specific embodiments, the combined expression of the siliencing element and the suppressor enhancer element increases the concentration of the inhibitory RNA in the plant cell, plant, plant part, plant tissue or phloem over the level that is achieved when the silencing element is expressed alone. As used herein, an “increased level of inhibitory RNA” comprises any statistically significant increase in the level of RNAi produced in a plant having the combined expression when compared to an appropriate control plant. For example, an increase in the level of RNAi in the plant, plant part or the plant cell can comprise at least about a 1%, about a 1%-5%, about a 5%-10%, about a 10%-20%, about a 20%-30%, about a 30%-40%, about a 40%-50%, about a 50%-60%, about 60-70%, about 70%-80%, about a 80%-90%, about a 90%-100% or greater increase in the level of RNAi in the plant, plant part, plant cell, or phloem when compared to an appropriate control. In other embodiments, the increase in the level of RNAi in the plant, plant part, plant cell, or phloem can comprise at least about a 1 fold, about a 1 fold-5 fold, about a 5 fold-10 fold, about a 10 fold-20 fold, about a 20 fold-30 fold, about a 30 fold-40 fold, about a 40 fold-50 fold, about a 50 fold-60 fold, about 60 fold-70 fold, about 70 fold-80 fold, about a 80 fold-90 fold, about a 90 fold-100 fold or greater increase in the level of RNAi in the plant, plant part, plant cell or phloem when compared to an appropriate control. Methods to assay for an increase in the level of RNAi are discussed elsewhere herein.

A “double stranded RNA silencing element” or “dsRNA” comprises at least one transcript that is capable of forming a dsRNA. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of least two distinct RNA strands. The dsRNA molecule(s) employed in the methods and compositions of the invention mediate the reduction of expression of a target sequence, for example, by mediating RNA interference “RNAi” or gene silencing in a sequence-specific manner. In specific embodiments, the dsRNA is capable of reducing or eliminating the level or expression of a target polynucleotide or the polypeptide encoded thereby in a pest.

The dsRNA can reduce or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, for example, Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional iRNA that are capable of reducing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (siRNA), double-stranded RNA (dsRNA), micro-RNA (miRNA), hairpin RNA, short hairpin RNA (shRNA), post-transcriptional gene silencing RNA (ptgsRNA), and others.

In specific embodiments, at least one strand of the duplex or double-stranded region of the dsRNA shares sufficient sequence identity or sequence complementarity to the target polynucleotide to allow for the dsRNA to reduce the level of expression of the target sequence. As used herein, the strand that is complementary to the target polynucleotide is the “antisense strand” and the strand homologous to the target polynucleotide is the “sense strand.”

In one embodiment, the dsRNA comprises a hairpin RNA. A hairpin RNA comprises an RNA molecule that is capable of folding back onto itself to form a double stranded structure. Multiple structures can be employed as hairpin elements. In specific embodiments, the dsRNA suppression element comprises a hairpin element which comprises in the following order, a first segment, a second segment, and a third segment, where the first and the third segment share sufficient complementarity to allow the transcribed RNA to form a double-stranded stem-loop structure.

The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., segments 1 and 2 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication No. WO 02/00904, herein incorporated by reference. In specific embodiments, the loop region can be optimized to be as short as possible while still providing enough intramolecular flexibility to allow the formation of the base-paired stem region. Accordingly, the loop sequence is generally less than 1000, 900, 800, 700, 600, 500, 400, 300, 200, 100, 50, 25, 20, 15, 10 nucleotides or less.

The “first” and the “third” segment of the hairpin RNA molecule comprise the base-paired stem of the hairpin structure. The first and the third segments are inverted repeats of one another and share sufficient complementarity to allow the formation of the base-paired stem region. In specific embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the third segment are at least about 1000, 500, 400, 300, 200, 100, 50, 40, 30, 25, 20, 15 or 10 nucleotides in length. In specific embodiments, the length of the first and/or the third segment is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 20 nucleotides. In other embodiments, the length of the first and/or the third segment comprises at least 10-20 nucleotides, 20-35 nucleotides, 30-45 nucleotides, 40-50 nucleotides, 50-100 nucleotides, or 100-300 nucleotides. See, for example, International Publication No. WO 0200904. In specific embodiments, the first and the third segment comprises at least 20 nucleotides having at least 85% complementary to the first segment. In still other embodiments, the first and the third segments which form the stem-loop structure of the hairpin comprises 3′ or 5′ overhang regions having unpaired nucleotide residues.

In specific embodiments, the sequences used in the first, the second, and/or the third segments comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the silencing element. Thus, in some embodiments of the invention, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000, or more than 1000 nucleotides that share sufficient sequence identity to the target polynucleotide to allow for a decrease in expression levels of the target polynucleotide when expressed in an appropriate cell. In other embodiments, the domain is between about 15 to 50 nucleotides, about 20-35 nucleotides, about 25-50 nucleotides, about 20 to 75 nucleotides, about 40-90 nucleotides about 15-100 nucleotides.

In specific embodiments, the domain of the first, the second, and/or the third segment has 100% sequence identity to the target polynucleotide. In other embodiments, the domain of the first, the second and/or the third segment having homology to the target polypeptide have at least 50%, 60%, 70%, 80%, 85%, 90%, 91%, 92%, 93%, 94%, 95%, 96%, 97%, 98%, 99%, or greater sequence identity to a region of the target polynucleotide. The sequence identity of the domains of the first, the second and/or the third segments to the target polynucleotide need only be sufficient to decrease expression of the target polynucleotide of interest. See, for example, Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

The amount of complementarity shared between the first, second, and/or third segment and the target polynucleotide or the amount of complementarity shared between the first segment and the third segment (i.e., the stem of the hairpin structure) may vary depending on the organism in which gene expression is to be controlled. Some organisms or cell types may require exact pairing or 100% identity, while other organisms or cell types may tolerate some mismatching. In some cells, for example, a single nucleotide mismatch in the targeting sequence abrogates the ability to suppress gene expression. In these cells, the suppression cassettes of the invention can be used to target the suppression of mutant genes, for example, oncogenes whose transcripts comprise point mutations and therefore they can be specifically targeted using the methods and compositions of the invention without altering the expression of the remaining wild-type allele.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In specific embodiments a domain of the silencing element shares sufficient homology to at least about 15 consecutive nucleotides from about nucleotides 1-50, 50-100, 100-150, 150-200, 200-250, 250-300, 300-350, 350-400, 400-450, 450-500, 550-600, 600-650, 650-700, 750-800, 850-900, 950-1000, 1000-1050, 1050-1100, 1100-1200, 1200-1300, 1300-1400, 1400-1500, 1500-1600, 1600-1700, 1700-1800, 1800-1900, 1900-2000 of the target sequence. In some instances to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al. (2003) J. Biol. Chem. 278:7108-7118 and Yang et al. (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

The hairpin silencing element may also be designed such that the sense sequence or the antisense sequence do not correspond to a target polynucleotide. In this embodiment, the sense and antisense sequence flank a loop sequence that comprises a nucleotide sequence corresponding to all or part of the target polynucleotide. Thus, it is the loop region that determines the specificity of the RNA interference. See, for example, WO 02/00904, herein incorporated by reference.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al. (2002) PNAS 99 (Suppl. 4):16499-16506 and Mette et al. (2000) EMBO J. 19(19):5194-5201.

In other embodiments, the dsRNA can comprise a small RNA (sRNA). sRNAs can comprise both micro RNA (miRNA) and short-interfering RNA (siRNA) (Meister and Tuschl (2004) Nature 431:343-349 and Bonetta et al. (2004) Nature Methods 1:79-86). miRNAs are regulatory agents comprising about 19 ribonucleotides which are highly efficient at inhibiting the expression of target polynucleotides. See, for example Javier et al. (2003) Nature 425: 257-263, herein incorporated by reference. For miRNA interference, the silencing element can be designed to express a dsRNA molecule that forms a hairpin structure containing a 19-nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence.

When expressing an miRNA, it is recognized that various forms of an miRNA can be transcribed including, for example, the primary transcript (termed the “pri-miRNA”) which is processed through various nucleolytic steps to a shorter precursor miRNA (termed the “pre-miRNA”); the pre-miRNA; or the final (mature) miRNA is present in a duplex, the two strands being referred to as the miRNA (the strand that will eventually basepair with the target) and miRNA*. The pre-miRNA is a substrate for a form of dicer that removes the miRNA/miRNA* duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

The methods and compositions of the invention employ silencing elements that when transcribed “form” a dsRNA molecule. Accordingly, the heterologous polynucleotide being expressed need not form the dsRNA by itself, but can interact with other sequences in the cell or, in specific embodiments, the pest gut after ingestion to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then reduce the level of expression of the gene or genes to be silenced. See, for example, U.S. Provisional Application No. 60/691,613, filed Jun. 17, 2005, entitled “Methods and Compositions for Gene Silencing, herein incorporated by reference. The construct can be designed to have a target for an endogenous miRNA or alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

### V. Variants and Fragments

By “fragment” is intended a portion of the polynucleotide or a portion of the amino acid sequence and hence protein encoded thereby. Fragments of a polynucleotide may encode protein fragments that retain the biological activity of the native protein. Alternatively, fragments of a polynucleotide that are useful as a silencing element or as a suppressor enhancer element do not need to encode proteins that retain biological activity. Thus, fragments of a nucleotide sequence may range from at least about 10, about 15, about 20 nucleotides, about 50 nucleotides, about 75 nucleotides, about 100 nucleotides, about 200 nucleotides, about 300 nucleotides, about 400 nucleotides, about 500 nucleotides, about 600 nucleotides, about 700 nucleotides and up to the full-length polynucleotide employed in the invention. Methods to assay for the activity of a desired silencing element or suppressor enhancer element are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the polypeptides employed in the invention. Variant polynucleotides also include synthetically derived polynucleotide, such as those generated, for example, by using site-directed mutagenesis, but continue to retain the desired activity. Generally, variants of a particular polynucleotide of the invention (i.e., a silencing element) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides employed in the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, as discussed elsewhere herein. Such variants may result from, for example, genetic polymorphism or from human manipulation. Biologically active variants of a native protein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

### VI. DNA Constructs

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotide encoding the silencing element and/or suppressor enhancer element employed in the methods and compositions of the invention can be provided in an expression cassette for expression in a plant or organism of interest. It is recognized that multiple silencing elements and/or suppressor enhancer elements can be used. For example, multiple identical silencing elements and/or multiple identical suppressor enhancer elements; multiple silencing elements targeting different regions of the target sequence and/or multiple suppressor enhancer elements targeting different regions of the target sequence; multiple silencing elements from different target sequences and/or multiple suppressor enhancer elements from different target sequences can be used.

It is recognized that each silencing element can be contained in a single or separate cassette, DNA construct, or vector. Similarly, one or more polynucleotide comprising the suppressor enhancer element can be on a single or on multiple constructs or vectors. Likewise, the two elements (i.e., the silencing element and the suppressor enhancer element can be found on separate DNA constructs and/or vectors or alternatively be contained on the same construct and/or vector. As discussed, any means of providing the silencing element and/or the suppressor enhancer sequence is contemplated. A host cell, such as a plant or plant cell, can be transformed with a single cassette comprising DNA encoding one or more silencing elements and/or suppressor enhancer element or separate cassettes comprising each silencing element and/or suppressor enhancer element can be used to transform a plant or plant cell or host cell. Likewise, a host cell or a plant transformed with one component can be subsequently transformed with the second component. One or more silencing elements and/or suppressor enhancer element can also be brought together by sexual crossing. That is, a first plant comprising one component is crossed with a second plant comprising the second component. Progeny plants from the cross will comprise both components.

An expression cassette can include 5′ and 3′ regulatory sequences operably linked to the polynucleotide of the invention. “Operably linked” is intended to mean a functional linkage between two or more elements. For example, an operable linkage between a polynucleotide of the invention and a regulatory sequence (i.e., a promoter) is a functional link that allows for expression of the polynucleotide of the invention. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a polynucleotide comprising the silencing element and/or a suppressor enhancer element, and a transcriptional and translational termination region (i.e., termination region) functional in plants. The regulatory regions (i.e., promoters, transcriptional regulatory regions, and translational termination regions) and/or the polynucleotides employed in the invention may be native/analogous to the host cell or to each other. Alternatively, the regulatory regions and/or the polynucleotide employed in the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide encoding the silencing element, may be native with the plant host, or may be derived from another source (i.e., foreign or heterologous) to the promoter, the polynucleotide comprising silencing element, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The polynucleotide encoding the silencing element can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

An inducible promoter, for instance, a pathogen-inducible promoter could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant. Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Nail. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

In one embodiment of this invention the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells.

Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters in accordance with this invention include but are not limited to the promoters selected from the group consisting of: the SCSV3, SCSV4, SCSV5, and SCSV7 promoters (Schunmann et al. (2003) Plant Functional Biology 30:453-60; the rolC gene promoter of Agrobacterium rhizogenes(Kiyokawa et al. (1994) Plant Physiology 104:801-02; Pandolfini et al. (2003) BioMedCentral (BMC) Biotechnology 3:7, (www.biomedcentral.com/1472-6750/3/7); Graham et al. (1997) Plant Mol. Biol. 33:729-35; Guivarc′h et al. (1996); Almon et al. (1997) Plant Physiol. 115:1599-607; the rolA gene promoter of Agrobacterium rhizogenes (Dehio et al. (1993) Plant Mol. Biol. 23:1199-210); the promoter of the Agrobacterium tumefaciens T-DNA gene 5 (Korber et al. (1991) EMBO J. 10:3983-91); the rice sucrose synthase RSs1 gene promoter (Shi et al. (1994) J. Exp. Bot. 45:623-31); the CoYMV or Commelina yellow mottle badnavirus promoter (Medberry et al. (1992) Plant Cell 4:185-92; Zhou et al. (1998) Chin. J. Biotechnol. 14:9-16); the CFDV or coconut foliar decay virus promoter (Rohde et al. (1994) Plant Mol. Biol. 27:623-28; Hehn and Rhode (1998) J. Gen. Virol. 79:1495-99); the RTBV or rice tungro bacilliform virus promoter (Yin and Beachy (1995) Plant J. 7:969-80; Yin et al. (1997) Plant J. 12:1179-80); the pea glutamin synthase GS3A gene (Edwards et al. (1990) Proc. Natl. Acad. Sci. USA 87:3459-63; Brears et al. (1991) Plant J. 1:235-44); the inv CD111 and inv CD141 promoters of the potato invertase genes (Hedley et al. (2000) J. Exp. Botany 51:817-21); the promoter isolated from Arabidopsis shown to have phloem-specific expression in tobacco by Kertbundit et al. (1991) Proc. Natl. Acad. Sci. USA 88:5212-16); the VAHOX1 promoter region (Tornero et al. (1996) Plant J. 9:639-48); the pea cell wall invertase gene promoter (Zhang et al. (1996) Plant Physiol. 112:1111-17); the promoter of the endogenous cotton protein related to chitinase of US published patent application 20030106097, an acid invertase gene promoter from carrot (Ramloch-Lorenz et al. (1993) The Plant J. 4:545-54); the promoter of the sulfate transporter geneSultr1; 3 (Yoshimoto et al. (2003) Plant Physiol. 131:1511-17); a promoter of a sucrose synthase gene (Nolte and Koch (1993) Plant Physiol. 101:899-905); and the promoter of a tobacco sucrose transporter gene (Kuhn et al. (1997) Science 275-1298-1300).

Possible promoters also include the Black Chemy promoter for Prunasin Hydrolase (PH DL1.4 PRO) (U.S. Pat. No. 6,797,859), Thioredoxin H promoter from cucumber and rice (Fukuda A et al. (2005). Plant Cell Physiol. 46(11): 1779-86), Rice (RSs1) (Shi, T. Wang et al. (1994). J. Exp. Bot. 45(274): 623-631) and maize sucrose synthese-1 promoters (Yang., N-S. et al. (1990) PNAS 87:4144-4148), PP2 promoter from pumpkin Guo, H. et al. (2004) Transgenic Research 13:559-566), At SUC2 promoter (Truernit, E. et al. (1995) Planta 196(3):564-70., At SAM-1 (S-adenosylmethionine synthetase) (Mijnsbrugge K V. et al. (1996) Planr. Cell. Physiol. 37(8): 1108-1115), and the Rice tungro bacilliform virus (RTBV) promoter (Bhattacharyya-Pakrasi et al. (1993) Plant J. 4(1):71-79).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16.215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used in the present invention.

### VII. Various Compositions Comprising Silencing Elements

In one example, a plant or a host cell is transformed with a DNA construct or expression cassette for expression of at least one silencing element and/or the expression of the suppressor enhancer element. It is recognized that the composition can comprise a cell (such as plant cell or a bacterial cell), in which a polynucleotide encoding the silencing element and the polynucleotide comprising the suppressor enhancer element is stably incorporated into the genome and operably linked to promoters active in the cell.

It is recognized that the polynucleotides comprising sequences encoding the silencing element and the suppressor enhancer element can be used to transform organisms to provide for host organism production of these components, and subsequent application of the host organism to the environment of the target pest(s). Such host organisms include baculoviruses, bacteria, and the like. In this manner, the combination of polynucleotides encoding the silencing element may be introduced via a suitable vector into a microbial host, and said host applied to the environment, or to plants or animals.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be stably incorporated into the genome of the cell (e.g., chromosome, plasmid, plastid, or mitochondrial DNA), converted into an autonomous replicon, or transiently expressed (e.g., transfected mRNA).

Microorganism hosts that are known to occupy the “phytosphere” (phylloplane, phyllosphere, rhizosphere, and/or rhizoplana) of one or more crops of interest may be selected. These microorganisms are selected so as to be capable of successfully competing in the particular environment with the wild-type microorganisms, provide for stable maintenance and expression of the sequences encoding the silencing element and the target sequence, and desirably, provide for improved protection of the components from environmental degradation and inactivation.

Such microorganisms include bacteria, algae, and fungi. Of particular interest are microorganisms such as bacteria, e.g., Pseudomonas, Ervinia, Serratia, Klebsiella, Xanthomonas, Streptomyces, Rhizobium, Rhodopseudomonas, Methylius, Agrobacterium, Acetobacter, Lactobacillus, Arthrobacter, Azotobacter, Leuconostoc, and Alcaligenes, fungi, particularly yeast, e.g., Saccharomyces, Cryptococcus, Kluyveromyces, Sporobolomyces, Rhodotorula, and Aureobasidium. Of particular interest are such phytosphere bacterial species as Pseudomonas syringae, Pseudomonasfluorescens, Serratia marcescens, Acetobacter xylinum, Agrobacteria, Rhodopseudomonas spheroides, Xanthomonas campestris, Rhizobium melioti, Alcaligenes entrophus, Clavibacter xyli and Azotobacter vinlandir, and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces rosues, S. odorus, Kluyveromyces veronae, and Aureobasidium pollulans. Of particular interest are the pigmented microorganisms.

A number of ways are available for introducing the polynucleotide comprising the silencing element and/or the suppressor enhancer element into the microorganism host under conditions that allow for stable maintenance and expression of such nucleotide sequences. For example, expression cassettes can be constructed which include the nucleotide constructs of interest operably linked with the transcriptional and translational regulatory signals for expression of the nucleotide constructs, and a nucleotide sequence homologous with a sequence in the host organism, whereby integration will occur, and/or a replication system that is functional in the host, whereby integration or stable maintenance will occur.

Transcriptional and translational regulatory signals include, but are not limited to, promoters, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (2000); Molecular Cloning. A Laboratory Manual (3^{rd} ed.; Cold Spring Harbor Laboratory Press, Plainview, N.Y.); Davis et al. (1980) Advanced Bacterial Genetics (Cold Spring Harbor Laboratory, Cold Spring Harbor, N.Y.; and the references cited therein.

Suitable host cells include the prokaryotes and the lower eukaryotes, such as fungi. Illustrative prokaryotes, both Gram-negative and Gram-positive, include Enterobacteriaceae, such as Escherichia, Erwinia, Shigella, Salnionella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like.

Characteristics of particular interest in selecting a host cell for purposes of the invention include ease of introducing the coding sequence into the host, availability of expression systems, efficiency of expression, stability in the host, and the presence of auxiliary genetic capabilities. Characteristics of interest for use as a pesticide microcapsule include protective qualities, such as thick cell walls, pigmentation, and intracellular packaging or formation of inclusion bodies; leaf affinity; lack of mammalian toxicity; attractiveness to pests for ingestion; and the like. Other considerations include ease of formulation and handling, economics, storage stability, and the like.

Host organisms of particular interest include yeast, such as Rhodotorula spp., Aureobasidium spp., Saccharomyces spp., and Sporobolomyces spp., phylloplane organisms such as Pseudomonas spp., Erwinia spp., and Flavobacterium spp., and other such organisms, including Pseudomonas aeruginosa, Pseudomonasfluorescens, Saccharomyces cerevisiae, Bacillus thuringiensis, Escherichia coli, Bacillus subtilis, and the like.

The sequences encoding the silencing elements and/or the suppressor enhancer element encompassed by the invention can be introduced into microorganisms that multiply on plants (epiphytes) to deliver these components to potential target pests. Epiphytes, for example, can be gram-positive or gram-negative bacteria.

The silencing element and/or the suppressor enhancer element can be fermented in a bacterial host and the resulting bacteria processed and used as a microbial spray in the same manner that Bacillus thuringiensis strains have been used as insecticidal sprays. Any suitable microorganism can be used for this purpose. Pseudomonas has been used to express Bacillus thuringiensis endotoxins as encapsulated proteins and the resulting cells processed and sprayed as an insecticide Gaertner et al. (1993), in Advanced Engineered Pesticides, ed. L. Kim (Marcel Decker, Inc.).

Alternatively, the components of the invention are produced by introducing heterologous genes into a cellular host. Expression of the heterologous sequences results, directly or indirectly, in the intracellular production of the silencing element and the target sequence. These compositions may then be formulated in accordance with conventional techniques for application to the environment hosting a target pest, e.g., soil, water, and foliage of plants. See, for example, EPA 0192319, and the references cited therein.

In the present invention, a transformed microorganism can be formulated with an acceptable carrier into separate or combined compositions that are, for example, a suspension, a solution, an emulsion, a dusting powder, a dispersible granule, a wettable powder, and an emulsifiable concentrate, an aerosol, an impregnated granule, an adjuvant, a coatable paste, and also encapsulations in, for example, polymer substances.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the present invention (i.e., at least one silencing element) are normally applied in the form of compositions and can be applied to the crop area, plant, or seed to be treated. For example, the compositions may be applied to grain in preparation for or during storage in a grain bin or silo, etc. The compositions may be applied simultaneously or in succession with other compounds. Methods of applying an active ingredient or a composition that contains at least one silencing element include, but are not limited to, foliar application, seed coating, and soil application. The number of applications and the rate of application depend on the intensity of infestation by the corresponding pest.

Suitable surface-active agents include, but are not limited to, anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitan fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions comprising the silencing element and the suppressor enhancer element can be in a suitable form for direct application or as a concentrate of primary composition that requires dilution with a suitable quantity of water or other dilutant before application.

The compositions (including the transformed microorganisms) can be applied to the environment of an insect pest by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the composition(s) and/or transformed microorganism(s) may be mixed with grain to protect the grain during storage. It is generally important to obtain good control of pests in the early stages of plant growth, as this is the time when the plant can be most severely damaged. The compositions can conveniently contain another insecticide if this is thought necessary. In an embodiment of the invention, the composition(s) is applied directly to the soil, at a time of planting, in granular form of a composition of a carrier and dead cells of a Bacillus strain or transformed microorganism of the invention. Another embodiment is a granular form of a composition comprising an agrochemical such as, for example, a herbicide, an insecticide, a fertilizer, in an inert carrier, and dead cells of a Bacillus strain or transformed microorganism of the invention.

IIX. Plants, Plant Parts, and Methods of Introducing Sequences into Plants

In one embodiment, the methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the silencing element sequence and/or the suppressor enhancer element can be provided to a plant using a variety of transient transformation methods. Such transient transformation methods include, but are not limited to, the introduction of the protein or variants and fragments thereof directly into the plant or the introduction of the transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, polynucleotides can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

In other embodiments, the silencer element and/or suppressor enhancer element may be introduced into plants by contacting plants with a virus or viral nucleic acids. Generally, such methods involve incorporating a nucleotide construct of the invention within a viral DNA or RNA molecule. Further, it is recognized that promoters of the invention also encompass promoters utilized for transcription by viral RNA polymerases.

Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In one embodiment, the plants, plant cells and plant parts having the combined expression of the silencing element with the suppressor enhancer element, or a fragment thereof, have an increased level of the inhibitory RNA produced from the silencing element over that achievable with only the expression of the silencing element alone.

In specific embodiments, systemic production of RNAi occurs throughout the entire plant. In further embodiments, the plant or plant parts of the invention have an improved loading of RNAi into the phloem when compared to a control expressing the silencing element construct alone and, thus provide better control of phloem feeding insects by an RNAi approach. In specific embodiments, the plants, plant parts, and plant cells of the invention can further be characterized as allowing for the production of a diverse population of RNAi species that can enhance the effectiveness of disrupting target gene expression.

### IX. Methods of Use

Methods for increasing the concentration of inhibitory RNA specific for a target sequence are provided. The method comprises the combined expression of the silencer element and the suppressor enhancer element in the cell. In specific embodiments, the method comprises introducing into a plant cell, a first polynucleotide comprising an inhibitory RNA precursor for a pest target sequence and a second polynucleotide comprising the suppressor enhancer element, wherein the combined expression of the silencer element and the suppressor enhancer element increases the concentration in the plant cell of an inhibitory RNA specific for the pest target sequence. In further embodiments, the combined expression of the silencing element and the suppressor enhancer element increases the concentration of an inhibitory RNA specific for the pest target sequence in the phloem of a plant comprising the plant cell.

Further provided are methods for controlling a pest comprising feeding to the pest a plant cell comprising a first polynucleotide comprising a silencing element for a pest target sequence and a second polynucleotide comprising the suppressor enhancer element. In specific embodiments, the combined expression of the silencer element and the suppressor enhancer element increases the concentration in the plant cell of an inhibitory RNA specific for the pest target sequence.

The pest can be fed the silencing element in a variety of ways. For example, in one embodiment, the polynucleotide comprising the silencing element and the suppressor enhancer element are introduced into a plant. As the pest feeds on the plant or part thereof expressing these sequences, the RNAi is delivered to the pest. When the silencing element and/or the suppressor enhancer element is delivered to the plant in this manner, it is recognized that either one or both of the polynucleotides can be expressed constitutively or alternatively, one or both may be produced in a stage-specific manner by employing the various inducible or tissue-preferred or developmentally regulated promoters that are discussed elsewhere herein. For example, the silencer element and the suppressor enhancer element could be expressed in aerial plant tissues such as, the leaves, stem, flower, etc. In other embodiments, the siliencing element and the suppressor enhancer element is expressed in a root. In these embodiments, Hemioptera such as grape phylloxera can be targeted.

In certain embodiments, the constructs of the present invention can be stacked with any combination of polynucleotide sequences of interest in order to create plants with a desired trait. A trait, as used herein, refers to the phenotype derived from a particular sequence or groups of sequences. For example, the polynucleotides of the present invention may be stacked with any other polynucleotides encoding polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like. The combinations generated can also include multiple copies of any one of the polynucleotides of interest. The polynucleotides of the present invention can also be stacked with any other gene or combination of genes to produce plants with a variety of desired trait combinations including, but not limited to, traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122) and high methionine proteins (Pedersen et al. (1986) J. Biol. Chem. 261:6279; Kirihara et al. (1988) Gene 71:359; and Musumura et al. (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)); the disclosures of which are herein incorporated by reference.

The polynucleotides of the present invention can also be stacked with traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference. One could also combine the polynucleotides of the present invention with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821); the disclosures of which are herein incorporated by reference.

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

The following examples are offered by way of illustration and not by way of limitation.

### EXPERIMENTAL

### Example 1

### Target Sequences, Silencing Elements, and Suppression Enhancers Elements

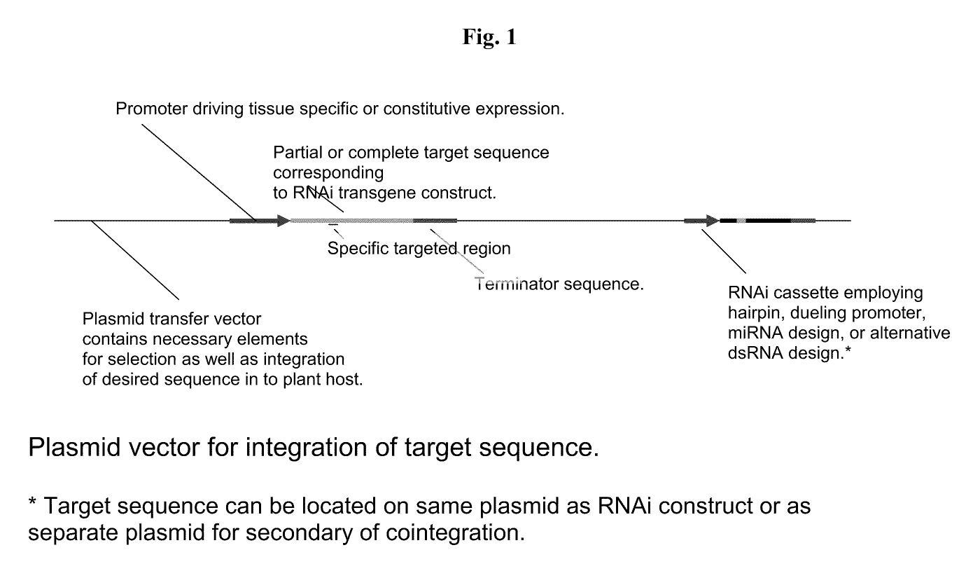

Disruption of insect gene function via RNAi can produce specific activity against target insects. This specificity is enhanced by delivery of the dsRNAs via transgenic plants. As described above, methods and compositions are provided which increase the level of RNAi through the combined expression of a suppressor enhancer element comprising the target sequence or a fragment or variant thereof and the silencing element. FIG. 1 provides non-limiting examples of full-length target sequences from Lepidoptera which can be used to design the appropriate suppressor enhancer element and/or silencing element for combined expression. Table I provides non-limiting examples of primers and their respective target sequences that can be used in the methods of the invention.

In specific embodiments, the suppressor enhancer element comprises a portion or the entire sequence which is desired to suppress. For example, the sequences set forth in SEQ ID NOS:1-58. Such a suppressor sequence could be placed under the control of an appropriate regulatory element and could be part of the same or different transformation vector used to deliver the silencer element or as a second vector used to co-transform recipient plants or cells or to secondarily transform cells or plants previously transformed with the silencer element.

TABLE 1

(Note: the sense primer sequence and the antisense primer sequences shown in
table 1 were generated having 2 thymine residues at the 3′ end.)

| Pest | | | | SEQ ID (target seq: |
|---|---|---|---|---|
| sequence | target seq | primer 1 | primer 2 | primer 1: primer 2) |
| ise1c.pk0 | AACATGGTATCCGACTTCAGG | CAUGGUAUCCGACUUC | CCUGAAGUCGGAUACC | 59/60/61 |
| 02.m13 | AA | AGG | AUG | |
| | AAGGTCGCTGACGAGAACAAG | GGUCGCUGACGAGAAC | CUUGUUCUCGUCAGCG | 62/63/64 |
| | GA | AAG | ACC | |
| | AAGTGTCCTGGGCTTGAGTTC | GUGUCCUGGGCUUGAG | GAACUCAAGCCCAGGA | 65/66/67 |
| | CA | UUC | CAC | |
| ise1c.pk0 | AAGAAGAAGCTCCTCCACGTG | GAAGAAGCUCCUCCAC | CACGUGGAGGAGCUUC | 68/69/70 |
| 03.f7 | TT | GUG | UUC | |
| | AAGGTCGCTGACGAGAACAAG | GGUCGCUGACGAGAAC | CUUGUUCUCGUCAGCG | 71/72/73 |
| | GA | AAG | ACC | |
| | AATGTCCTGGGGCTGAGTTTC | UGUCCUGGGGCUGAGU | GAAACUCAGCCCCAGG | 74/75/76 |
| | AA | UUC | ACA | |
| ise1c.pk0 | AAGAATAAGCTCCTCCACGTG | GAAUAAGCUCCUCCAC | CACGUGGAGGAGCUUA | 77/78/79 |
| 05.a15 | TT | GUG | UUCtt | |
| | AATTTGTCGAGGAGACCCTAT | UUUGUCGAGGAGACCC | AUAGGGUCUCCUCGAC | 80/81/82 |
| | TG | UAU | AAA | |
| | AAGTTCGCGTTCACTCTTGAA | GUUCGCGUUCACUCUU | UUCAAGAGUGAACGCG | 83/84/85 |
| | GA | GAA | AAC | |
| ise1c.pk0 | AACTGCCCCTTAACCTCATCT | CUGCCCCUUAACCUCA | AGAUGAGGUUAAGGGG | 86/87/88 |
| 06.d24 | AT | UCU | CAG | |
| | AATCACGCTGAAACCACTGTA | UCACGCUGAAACCACU | UACAGUGGUUUCAGCG | 89/90/91 |
| | TA | GUA | UGA | |
| ise2c.pk0 | AAAATATGGCGCGCCTATTGT | AAUAUGGCGCGCCUAU | ACAAUAGGCGCGCCAU | 92/93/94 |
| 09.i4 | TT | UGUtt | AUU | |
| | AACGTTCTCGGTCTTTCACTG | CGUUCUCGGUCUUUCA | CAGUGAAAGACCGAGA | 95/96/97 |
| | CT | CUGtt | ACG | |
| | AAGTCATCGTTCCAAGTCTAC | GUCAUCGUUCCAAGUC | GUAGACUUGGAACGAU | 98/99/100 |
| | CT | UAC | GAC | |
| ise2c.pk0 | AACCCCTTGAATGTTAAGGTC | CCCCUUGAAUGUUAAG | GACCUUAACAUUCAAG | 101/102/103 |
| 01.d19 | GG | GUC | GGG | |
| | AAGTACACCATGTTGCAAGTA | GUACACCAUGUUGCAA | UACUUGCAACAUGGUG | 104/105/106 |
| | TG | GUA | UAC | |
| | AACGTGTCCATGATGGCTGAC | CGUGUCCAUGAUGGCU | GUCAGCCAUCAUGGAC | 107/108/109 |
| | TC | GAC | ACG | |
| ise2c.pk0 | AAACCTACAAAATGGCCGAAA | ACCUACAAAAUGGCCG | UUUCGGCCAUUUUGUA | 110/111/112 |
| 01.e14 | AC | AAA | GGU | |
| | AATCTACGGACCCTTCTTTGG | UCUACGGACCCUUCUU | CCAAAGAAGGGUCCGU | 113/114/115 |
| | AG | UGG | AGA | |
| ise2c.pk0 | AACTCTGACGTCATCATCTAC | CUCUGACGUCAUCAUC | GUAGAUGAUGACCUCA | 116/117/118 |
| 01.f20 | GT | UAC | GAG | |
| | AAGTGCTTGGGTAACCCCGAC | GUGCUUGGGUAACCCC | GUCGGGGUUACCCAAG | 119/120/121 |
| | AG | GAC | CAC | |
| | AACTGGCTCATCTCCTACAGC | CUGGCUCAUCUCCUAC | GCUGUAGGAGAUGAGC | 122/123/124 |
| | AA | AGC | CAG | |
| ise2c.pk0 | AAACAGTGCGTCGTAATATAT | ACAGUGCGUCGUAAUA | AUAUAUUACGACGCAC | 125/126/127 |
| 10.h3 | TC | UAU | UGU | |
| | AAGGCACATGGTCCTTCACTG | GGCACAUGGUCCUUCA | CAGUGAAGGACCAUGU | 128/129/130 |
| | AT | CUG | GCC | |
| | AACACCATGACCCTCGTGTAC | CACCAUGACCCUCGUG | GUACACGAGGGUCAUG | 131/132/133 |
| | AA | UAC | GUG | |
| ise2c.pk0 | AACGAGGCCGGATCTCTTAAG | CGAGGCCGGAUCUCUU | CUUAAGAGAUCCGGCC | 134/135/136 |
| 07.k24 | CA | AAG | UCG | |
| | AACTTCACACATAACTAGACA | CUUCACACAUAACUAG | UGUCUAGUUAUGUGUG | 137/138/139 |
| | AA | ACA | AAG | |
| | AATGCGTGGCGATTTCAAACT | UUAGAAAUUAUAAGCC | CUGGGCUUAUAAUUUC | 140/141/142 |
| | TA | CAG | UAA | |
| ise2c.pk0 | AAAAAACACAGACCACGTTCA | AAAACACAGACCACGU | UGAACGUGGUCUGUGU | 143/144/145 |
| 11.a10 | CA | UCA | UUU | |
| | AATCGATGGTGGTGTTATTCG | UCGAUGGUGGUGUUAU | CGAAUAACACCACCAU | 146/147/148 |
| | CT | UCG | CGA | |
| ise2c.pk0 | AAAGAAAATGCTACGCGTTAC | AGAAAAUGCUACGCGU | GUAACGCGUAGCAUUU | 149/150/151 |
| 11.h12 | GA | UAC | UCU | |
| | AACCCTTGGACACTACTGGAA | CCCUUGGACACUACUG | UUCCAGUAGUGUCCAA | 152/153/154 |
| | GA | GAA | GGG | |
| | AAGGATCCTATGTGTACCAGG | GGAUCCUAUGUGUACC | CCUGGUACACAUAGGA | 155/156/157 |
| | TT | AGG | UCC | |
| ise2c.pk0 | AAACTCGGCACACAACACAAT | ACUCGGCACACAACAC | AUUGUGUUGUGUGCCG | 158/159/160 |
| 01.d22 | GG | AAU | AGU | |
| | AATACGAAGATATCTGCCCTT | UACGAAGAUAUCUGCC | AAGGGCAGAUAUCUUC | 161/162/163 |
| | CC | CUU | GUA | |
| | AATCAACAGCTCTTACATAAA | UCAACAGCUCUUACAU | UUUAUGUAAGAGCUGU | 164/165/166 |
| | TG | AAA | UGA | |
| ise2c.pk0 | AAAGAAGATCAGAAGATTGGC | AGAAGAUCAGAAGAUU | GCCAAUCUUCUGAUCU | 167/168/169 |
| 01.d9 | CG | GGC | UCU | |
| | AAAAGCCGTCTGCTATCCAAC | AAGCCGUCUGCUAUCC | GUUGGAUAGCAGACGG | 170/171/172 |
| | AA | AAC | CUU | |
| | AATGCTAAATGCCATGCTTGC | UGCUAAAUGCCAUGCU | GCAAGCAUGGCAUUUA | 173/174/175 |
| | AT | UGC | GCA | |
| ise2c.pk0 | AAGATCAGAAGATTGGCCGGA | GAUCAGAAGAUUGGCC | UCCGGCCAAUCUUCUG | 176/177/178 |
| 01.i23 | AG | GGA | AUC | |
| | AATTCTTCAGCAAATCGATAC | UUCUUCAGCAAAUCGA | GUAUCGAUUUGCUGAA | 179/180/181 |
| | CA | UAC | GAA | |
| | AAATGCTGTCAAGAGGATTTA | AUGCUGUCAAGAGGAU | UAAAUCCUCUUGACAG | 182/183/184 |
| | AA | UUA | CAU | |
| ise2c.pk0 | AAGCTCGAGACTTGCTCTTGA | GCUCGAGACUUGCUCU | UCAAGAGCAAGUCUCG | 185/186/187 |
| 01.124 | TG | UGA | AGC | |
| | AACTGTTAGCTCAAGGTCTGC | CUGUUAGCUCAAGGUC | GCAGACCUUGAGCUAA | 188/189/190 |
| | TA | UGC | CAG | |
| | AAGACTTTCTATCAGAATTTG | GACUUUCUAUCAGAAU | CAAAUUCUGAUAGAAA | 191/192/193 |
| | CG | UUG | GUC | |
| ise2c.pk0 | AAACTTAATCATGGACGACGA | ACUUAAUCAUGGACGA | UCGUCGUCCAUGAUUA | 194/195/196 |
| 05.b9 | CA | CGA | AGU | |
| | AAAGAAGAAGAAGAAGAAGGG | AGAAGAAGAAGAAGAA | CCCUUCUUCUUCUUCU | 197/198/199 |
| | AG | GGG | UCU | |
| | AAGATCAAGAGAATGTCGAGG | GAUCAAGAGAAUGUCG | CCUCGACAUUCUCUUG | 200/201/202 |
| | AT | AGG | AUC | |
| ise2c.pk0 | AAAATCGTCGGTTTTAGCGAC | AAUCGUGGCUUUUAGC | GUCGCUAAAACCGACG | 203/204/205 |
| 02.m10 | GT | GAC | AUU | |
| | AACTGTCAATAGGCAGTATGC | CUGUCAAUAGGCAGUA | GCAUACUGCCUAUUGA | 206/207/208 |
| | GT | UGC | CAG | |
| | AACCTGTACCAACAGACCACT | CCUGUACCAACAGACC | AGUGGUCUGUUGGUAC | 209/210/211 |
| | GG | ACU | AGG | |
| ise2c.pk0 | AACCAAAAATGGGCAAGGAAA | CCAAAAAUGGGCAAGG | UUUCCUUGCCCAUUUU | 212/213/214 |
| 01.c14 | AG | AAA | UGG | |
| | AACGTGGTATCACCATCGATA | CGUGGUAUCACCAUCG | UAUCGAUGGUGAUACC | 215/216/217 |
| | TT | AUA | ACG | |
| | AACAAAATGGAGTCCACTGAG | CAAAAUGGACUCCACU | CUCAGUGGAGUCCAUU | 218/219/220 |
| | CC | GAG | UUG | |
| ise2c.pk0 | AATCCGTGACTAACCAAAAAT | UCCGUGACUAACCAAA | AUUUUUGGUUAGUCAC | 221/222/223 |
| 01.d16 | GG | AAU | GGA | |
| | AACATTGTCGTCATTGGACAC | CAUUGUCGUCAUUGGA | GUGUCCAAUGACGACA | 224/225/226 |
| | GT | CAC | AUG | |
| ise2c.pk0 | AATTTGTGAGACTGGTGGCCG | UUUGUGAGACUGGUGG | CGGCCACCAGUCUCAC | 227/228/229 |
| 05.h3 | AA | CCG | AAA | |
| | AATCTGATTGTATTCGCCCCC | UCUGAUUGUAUUCGCC | GGGGGCGAAUACAAUC | 230/231/232 |
| | TC | CCC | AGA | |
| | AACACTCTAGTTCTGCCTATT | CACUCUAGUUCUGCCU | AAUAGGCAGAACUAGA | 233/234/235 |
| | CT | AUU | GUG | |
| ise2c.pk0 | AACACACATCACAATGGCGGA | CACACAUCACAAUGGC | UCCGCCAUUGUGAUGU | 236/237/238 |
| 01.d21 | TA | GGA | GUG | |
| | AAGGATGGCATCATCGGCAAG | GGAUGGCAUCAUCGGC | CUUGCCGAUGAUGCCA | 239/240/241 |
| | AA | AAG | UCC | |
| | AAAGGCTTCATCGACACCGCG | AGGCUUCAUCGACACC | CGCGGUGUCGAUGAAG | 242/243/244 |
| | AA | GCG | CCU | |
| ise2c.pk0 | AAACTCCAATTATAACCTACT | ACUCCAAUUAUAACCU | AGUAGGUUAUAAUUGG | 245/246/247 |
| 01.j9 | AG | ACU | AGU | |
| | AAGTACAAGGATCTGATCGGC | GUACAAGGAUCUGAUC | GCCGAUCAGAUCCUUG | 248/249/250 |
| | AA | GGC | UAC | |
| | AAGACTTTCTTCATGTGGCCC | GACUUUCUUCAUGUGG | GGGCCACAUGAAGAAA | 251/252/253 |
| | AT | CCC | GUC | |
| ise2c.pk0 | AAACAAAGTATCGCCTACACC | ACAAAGUAUCGCCUAC | GGUGUAGGCGAUACUU | 254/255/256 |
| 02.f12 | GC | ACC | UGU | |
| | AATAGCGTCGATCTTCAACGA | UAGCGUCGAUCUUCAA | UCGUUGAAGAUCGACG | 257/258/259 |
| | CT | CGA | CUA | |
| ise2c.pk0 | AACTCATAGAGCTTGATGTGT | CUCAUAGAGCUUGAUG | ACACAUCAAGCUCUAU | 260/261/262 |
| 01.b14 | GG | UGU | GAG | |
| | AAGATGTGGATGACGTCACTG | GAUGUGGAUGACGUCA | CAGUGACGUCAUCCAC | 263/264/265 |
| | GT | CUG | AUC | |
| | AACCTTCCTGATTCTCTTCTG | CCUUCCUGAUUCUCUU | CAGAAGAGAAUCAGGA | 266/267/268 |
| | TG | CUG | AGG | |
| ise2c.pk0 | AACAGTGCTTGTGATAAGTGA | CAGUGCUUGUGAUAAG | UCACUUAUCACAAGCA | 269/270/271 |
| 03.f2 | AC | UGA | CUG | |
| | AAGTTAATGGTGACTGCCCTC | GUUAAUGGUGACUGCC | GAGGGCAGUCACCAUU | 272/273/274 |
| | GA | CUC | AAC | |
| | AATAAAGCGATGACCCCATAG | UAAAGCGAUGACCCCA | CUAUGGGGUCAUCGCU | 275/276/277 |
| | GA | UAG | UUA | |
| ise2c.pk0 | AAACGGTACTGCAGCAAAAAG | ACGGUACUGCAGCAAA | CUUUUUGCUGCAGUAC | 278/279/280 |
| 05.120 | AC | AAG | CGU | |
| | AAGCTGCATACTTCTTGGCTC | GCUGCAUACUUCUUGG | GAGCCAAGAAGUAUGC | 281/282/283 |
| | TC | CUC | AGC | |
| | AAATGTTTACAGAGACGCGAT | AUGUUUACAGAGACGC | AUCGCGUCUCUGUAAA | 284/285/286 |
| | GA | GAU | CAU | |
| ise2c.pk0 | AACGTCGATCTTACCGAGTTC | CGUCGAUCUUACCGAG | GAACUCGGUAAGAUCG | 287/288/289 |
| 01.di | CA | UUC | ACG | |
| ise2c.pk0 | AATTCAAAATGCGTGAGTGCA | UUCAAAAUGCGUGAGU | UGCACUCACGCAUUUU | 290/291/292 |
| 01.k6 | TC | GCA | GAA | |
| | AAATCGTAGACCTAGTCCTCG | AUCGUAGACCUAGUCC | CGAGGACUAGGUCUAC | 293/294/295 |
| | AC | UCG | GAU | |
| ise2c.pk0 | AAACTCAATTCAAAATGCGTG | ACUCAAUUCAAAAUGC | CACGCAUUUUGAAUUG | 296/297/298 |
| 01.12 | AG | GUG | AGU | |
| | AACTTATCACTGGTAAGGAAG | CUUAUCACUGGUAAGG | CUUCCUUACCAGUGAU | 299/300/301 |
| | AT | AAG | AAG | |
| ise2c.pk0 | AAGAGTTACGAACCGTCACCA | GAGUUACGAACCGUCA | UGGUGACGGUUCGUAA | 302/303/304 |
| 02.b4 | TA | CCA | CUC | |
| | AAACTTAGTCCGGATAATGAA | ACUUAGUCCGGAUAAU | UUCAUUAUCCGGACUA | 305/306/307 |
| | CC | GAA | AGU | |
| | AAGGCGATGTACGAGAACCTG | GGCGAUGUACGAGAAC | CAGGUUCUCGUACAUC | 308/309/310 |
| | TT | CUG | GCC | |
| ise2c.pk0 | AACGACAAGATGCTGAAGGAG | CGACAAGAUGCUGAAG | CUCCUUCAGCAUCUUG | 311/312/313 |
| 01.j16 | AC | GAG | UCG | |
| ise2c.pk0 | AAGATAAAGGTCGCGTGTGGA | GAUAAAGGUCGCGUGU | UCCACACGCGACCUUU | 314/315/316 |
| 06.h23 | CC | GGA | AUC | |
| | AATGTCAAGACTGATCCAAAC | UGUCAAGACUGAUCCA | GUUUGGAUCAGUCUUG | 317/318/319 |
| | AC | AAC | ACA | |
| | AACATTCGAGTCTGAACAGGT | CAUUCGAGUCUGAACA | ACCUGUUCAGACUCGA | 320/321/322 |
| | GG | GGU | AUG | |

The date appearing in table 2 was carried out as follows:

### Droplet Feeding Assays:

First instar larvae were fed the 21 bp dsRNA primer pairs (50 uMolar) in a 20% sucrose solution which also contained blue food coloring. The primers had 2 bp overhangs at either end. Primer sequence was based on genes from the internal fall armyworm cDNA library. The target genes were selected based on review of literature indicating they were perturbable targets in other species or on predictions of which genes would be essential to development or physiology of the insect. After two hours most larvae had consumed the solution. Nevertheless, only larvae with blue gastric tracts were transferred to clean artificial diet (no dsRNA was incorporated into the diet). Insects were calculated to have consumed approximately 200 nl of fluid. After 48 hours, the insects were examined for stunting in comparison to negative controls of sucrose fed insects and insects fed negative control Ambion primers.

### Injection Assays.

Neonate or second instar fall armyworm were injected with a variety of RNA primer pairs at a concentration of 2 ug/ul. Injections were carried out with the aid of a micromanipulator and micropipette using 30× magnification with a dissecting microscope. Approximately 200 nL were injected into each insect and injections were aimed at the hemocoel of the insect rather than the gastric tract. Injections of Ambion negative control primer pairs indicated nearly 100% survival. The one lone death was likely due to mechanical injury. These injection assays indicated strong insecticidal activities from the majority of primer pairs based on fall armyworm sequences tested at this concentration. 2 primers were then injected in a range of concentrations from 2 ug/ul to 0.125 ug/ul. Mortality was observed at the highest rates but at lower rates, stunting was also observed. The EC50 concentration was estimated to be approximately 0.6 ug/ul.

### Diet Based Topical Feeding Assay:

Assays were carried out with primer pair concentrations of 0.67 ug/ul. 5 ul of the samples were applied to 100 ul of diet, 4 observations/sample. The experiment was repeated 4 times. The insects were scored for stunting and mortality at 72 hours. While some primer pairs demonstrated inconsistent activity, several primer pairs demonstrated consistent activity in these assays. Inconsistency is likely due at least in part to inconsistent definitions of stunting by the scorer (me). We hope to resolve this problem by adopting the scanalyzer to score these plates in the future and set a strict criteria for stunting and necessary consistency across the 4 observations. In the mean time, I have made the criteria for stunting more stringent which should help eliminate marginal calls that might in one experimental replicate make the grade and not in another.

A dose response was performed for all primer pairs at 0.67, 0.33, and 0.16 ug/ul. Typical dose response data was observed and primer pairs could be distinguished based on EC50 data. Many of the primer pairs demonstrated activity at the highest rate. In several other instances, primer pairs had activity at the two highest rates but did not demonstrate activity at the lowest rate. In one instance, a primer pair had clear mortality at the two highest rates and stunting even at the 0.16 ug/ul rate. This data is summarized in the table below. Note: the sense primer sequence and the antisense primer sequences shown in table 2 were generated having 2 thymine residues at the 3′ end.

TABLE 2

| | | | | | | | | SEQ ID |
|---|---|---|---|---|---|---|---|---|
| Target | | Injection | Droplet | Topi- | | | | (target seq: |
| pest | | Mortality | Feeding | cal | | | | primer 1: |
| seqeunce | gene id | (%) | Result | assay | target seq | primer 1 | primer 2 | primer 2) |
| ise1c.pk0 | Juvenile | 100, 77 | Active | No | AACATGGTATC | CAUGGUAUCCGA | CCUGAAGUCGGAU | 59/60/61 |
| 02.m13 | hormone | | | | CGACTTCAGGA | CUUCAGG | ACCAUG | |
| | query | | | | A | | | |
| | | NT | NT | No | AAGGTCGCTGA | GGUCGCUGACGA | CUUGUUCUCGUCA | 62/63/64 |
| | | | | | CGAGAACAAGG | GAACAAG | GCGACC | |
| | | | | | A | | | |
| | | NT | NT | Active | AAGTGTCCTGG | GUGUCCUGGGCU | GAACUCAAGCCCA | 65/66/67 |
| | | | | | GCTTGAGTTCC | UGAGUUC | GGACAC | |
| | | | | | A | | | |
| ise1c.pk0 | Juvenile | NT | NT | No | AAGAAGAAGCT | GAAGAAGCUCCU | CACGUGGAGGAGC | 68/69/70 |
| 03.f7 | hormone | | | | CCTCCACGTGT | CCACGUG | UUCUUC | |
| | query | | | | T | | | |
| | | NT | NT | No | AAGGTCGCTGA | GGUCGCUGACGA | CUUGUUCUCGUCA | 71/72/73 |
| | | | | | CGAGAACAAGG | GAACAAG | GCGACC | |
| | | | | | A | | | |
| | | NT | NT | Active | AATGTCCTGGG | UGUCCUGGGGCU | GAAACUCAGCCCC | 74/75/76 |
| | | | | | GCTGAGTTTCA | GAGUUUC | AGGACA | |
| | | | | | A | | | |
| ise1c.pk0 | Juvenile | NT | NT | Active | AAGAATAAGCT | GAAUAAGCUCCU | CACGUGGAGGAGC | 77/78/79 |
| 05.a15 | hormone | | | | CCTCCACGTGT | CCACGUG | UUAUUC | |
| | query | | | | T | | | |
| | | NT | NT | No | AATTTGTCGAG | UUUGUCGAGGAG | AUAGGGUCUCCUC | 80/81/82 |
| | | | | | GAGACCCTATT | ACCCUAU | GACAAA | |
| | | | | | G | | | |
| | | 100, 50 | Active | Active | AAGTTCGCGTT | GUUCGCGUUCAC | UUCAAGAGUGAAC | 83/84/85 |
| | | | | | CACTCTTGAAG | UCUUGAA | GCGAAC | |
| | | | | | A | | | |
| ise1c.pk0 | Juvenile | NT | NT | Active | AACTGCCCCTT | CUGCCCCUUAAC | AGAUGAGGUUAAG | 86/87/88 |
| 06.d24 | hormone | | | | AACCTCATCTA | CUCAUCU | GGGCAG | |
| | query | | | | T | | | |
| | | 60 | NT | Active | AATCACGCTGA | UCACGCUGAAAC | UACAGUGGUUUCA | 89/90/91 |
| | | | | | AACCACTGTAT | CACUGUA | GCGUGA | |
| | | | | | A | | | |
| ise2c.pk0 | Juvenile | 65 | NT | No | AAAATATGGCG | AAUAUGGCGCGC | ACAAUAGGCGCGC | 92/93/94 |
| 09.i4 | hormone | | | | CGCCTATTGTTT | CUAUUGU | CAUAUU | |
| | query | | | | | | | |
| | | NT | NT | No | AACGTTCTCGG | CGUUCUCGGUCU | CAGUGAAAGACCG | 95/96/97 |
| | | | | | TCTTTCACTGCT | UUCACUG | AGAACG | |
| | | 80 | NT | Active | AAGTCATCGTT | GUCAUCGUUCCA | GUAGACUUGGAAC | 98/99/100 |
| | | | | | CCAAGTCTACC | AGUCUAC | GAUGAC | |
| | | | | | T | | | |
| ise2c.pk0 | vacuolar | 90, | NT | Active | AACCCCTTGAA | CCCCUUGAAUGU | GACCUUAACAUUC | 101/102/103 |
| 01.d19 | query | | | | TGTTAAGGTCG | UAAGGUC | AAGGGG | |
| | | | | | G | | | |
| | | NT | NT | No | AAGTACACCAT | GUACACCAUGUU | UACUUGCAACAUG | 104/105/106 |
| | | | | | GTTGCAAGTAT | GCAAGUA | GUGUAC | |
| | | | | | G | | | |
| | | 50, 100 | No | Active | AACGTGTCCAT | CGUGUCCAUGAU | GUCAGCCAUCAUG | 107/108/109 |
| | | | | | GATGGCTGACT | GGCUGAC | GACACG | |
| | | | | | C | | | |
| ise2c.pk0 | vacuolar | NT | NT | No | AAACCTACAAA | ACCUACAAAAUG | UUUCGGCCAUUUU | 110/111/112 |
| 01.e14 | query | | | | ATGGCCGAAAA | GCCGAAA | GUAGGU | |
| | | | | | C | | | |
| | | NT | NT | Active | AATCTACGGAC | UCUACGGACCCU | CCAAAGAAGGGUC | 113/114/115 |
| | | | | | CCTTCTTTGGA | UCUUUGG | CGUAGA | |
| | | | | | G | | | |
| ise2c.pk0 | vacuolar | 77 | NT | Active | AACTCTGACGT | CUCUGACGUCAU | GUAGAUGAUGACG | 116/117/118 |
| 01.f20 | query | | | | CATCATCTACG | CAUCUAC | UCAGAG | |
| | | | | | T | | | |
| | | 100 | NT | No | AAGTGCTTGGG | GUGCUUGGGUAA | GUCGGGGUUACCC | 119/120/121 |
| | | | | | TAACCCCGACA | CCCCGAC | AAGCAC | |
| | | | | | G | | | |
| | | NT | NT | No | AACTGGCTCAT | CUGGCUCAUCUC | GCUGUAGGAGAUG | 122/123/124 |
| | | | | | CTCCTACAGCA | CUACAGC | AGCCAG | |
| | | | | | A | | | |
| ise2c.pk0 | cadherin | NT | NT | NT | AAACAGTGCGT | ACAGUGCGUCGU | AUAUAUUACGACG | 125/126/127 |
| 10.h3 | query | | | | CGTAATATATT | AAUAUAU | CACUGU | |
| | | | | | C | | | |
| | | NT | NT | Active | AAGGCACATGG | GGCACAUGGUCC | CAGUGAAGGACCA | 128/129/130 |
| | | | | | TCCTTCACTGAT | UUCACUG | UGUGCC | |
| | | 100, 80 | Active | No | AACACCATGAC | CACCAUGACCCU | GUACACGAGGGUC | 131/132/133 |
| | | | | | CCTCGTGTACA | CGUGUAC | AUGGUG | |
| | | | | | A | | | |
| ise2c.pk0 | cuticle | NT | NT | No | AACGAGGCCGG | CGAGGCCGGAUC | CUUAAGAGAUCCG | 134/135/136 |
| 07.k24 | protein | | | | ATCTCTTAAGC | UCUUAAG | GCCUCG | |
| | | | | | A | | | |
| | | NT | NT | No | AACTTCACACA | CUUCACACAUAA | UGUCUAGUUAUGU | 137/138/139 |
| | | | | | TAACTAGACAA | CUAGACA | GUGAAG | |
| | | | | | A | | | |
| | | NT | NT | No | AATGCGTGGCG | UUAGAAAUUAUA | CUGGGCUUAUAAU | 141/141/142 |
| | | | | | ATTTCAAACTT | AGCCCAG | UUCUAA | |
| | | | | | A | | | |
| ise2c.pk0 | cuticle | NT | NT | Active | AAAAAACACAG | AAAACACAGACC | UGAACGUGGUCUG | 143/144/145 |
| 11.a10 | protein | | | | ACCACGTTCAC | ACGUUCA | UGUUUU | |
| | | | | | A | | | |
| | | NT | NT | Active | AATCGATGGTG | UCGAUGGUGGUG | CGAAUAACACCAC | 146/147/148 |
| | | | | | GTGTTATTCGCT | UUAUUCG | CAUCGA | |
| ise2c.pk0 | cuticle | NT | NT | No | AAAGAAAATGC | AGAAAAUGCUAC | GUAACGCGUAGCA | 149/150/151 |
| 11.h12 | protein | | | | TACGCGTTACG | GCGUUAC | UUUUCU | |
| | | | | | A | | | |
| | | NT | NT | Active | AACCCTTGGAC | CCCUUGGACACU | UUCCAGUAGUGUC | 152/153/154 |
| | | | | | ACTACTGGAAG | ACUGGAA | CAAGGG | |
| | | | | | A | | | |
| | | 100, 50 | Active | Active | AAGGATCCTAT | GGAUCCUAUGUG | CCUGGUACACAUA | 155/156/157 |
| | | | | | GTGTACCAGGT | UACCAGG | GGAUCC | |
| | | | | | T | | | |
| ise2c.pk0 | translation | NT | NT | Active | AAACTCGGCAC | ACUCGGCACACA | AUUGUGUUGUGUG | 158/159/160 |
| 01.d22 | initiation | | | | ACAACACAATG | ACACAAU | CCGAGU | |
| | factor | | | | G | | | |
| | | NT | NT | Active | AATACGAAGAT | UACGAAGAUAUC | AAGGGCAGAUAUC | 161/162/163 |
| | | | | | ATCTGCCCTTCC | UGCCCUU | UUCGUA | |
| | | NT | NT | Active | AATCAACAGCT | UCAACAGCUCUU | UUUAUGUAAGAGC | 164/165/166 |
| | | | | | CTTACATAAAT | ACAUAAA | UGUUGA | |
| | | | | | G | | | |
| ise2c.pk0 | translation | NT | NT | No | AAAGAAGATCA | AGAAGAUCAGAA | GCCAAUCUUCUGA | 167/168/169 |
| 01.d9 | initiation | | | | GAAGATTGGCC | GAUUGGC | UCUUCU | |
| | factor | | | | G | | | |
| | | NT | NT | Active | AAAAGCCGTCT | AAGCCGUCUGCU | GUUGGAUAGCAGA | 170/171/172 |
| | | | | | GCTATCCAACA | AUCCAAC | CGGCUU | |
| | | | | | A | | | |
| | | NT | NT | Active | AATGCTAAATG | UGCUAAAUGCCA | GCAAGCAUGGCAU | 173/174/175 |
| | | | | | CCATGCTTGCA | UGCUUGC | UUAGCA | |
| | | | | | T | | | |
| ise2c.pk0 | translation | NT | NT | Active | AAGATCAGAAG | GAUCAGAAGAUU | UCCGGCCAAUCUU | 176/177/178 |
| 01.i23 | initiation | | | | ATTGGCCGGAA | GGCCGGA | CUGAUC | |
| | factor | | | | G | | | |
| | | 100, 75 | Active | No | AATTCTTCAGC | UUCUUCAGCAAA | GUAUCGAUUUGCU | 179/180/181 |
| | | | | | AAATCGATACC | UCGAUAC | GAAGAA | |
| | | | | | A | | | |
| | | NT | NT | Active | AAATGCTGTCA | AUGCUGUCAAGA | UAAAUCCUCUUGA | 182/183/184 |
| | | | | | AGAGGATTTAA | GGAUUUA | CAGCAU | |
| | | | | | A | | | |
| ise2c.pk0 | translation | NT | NT | Active | AAGCTCGAGAC | GCUCGAGACUUG | UCAAGAGCAAGUC | 185/186/187 |
| 01.l24 | initiation | | | | TTGCTCTTGATG | CUCUUGAtt | UCGAGCtt | |
| | factor | | | | | | | |
| | | NT | NT | Active | AACTGTTAGCT | CUGUUAGCUCAA | GCAGACCUUGAGC | 188/189/190 |
| | | | | | CAAGGTCTGCT | GGUCUGC | UAACAG | |
| | | | | | A | | | |
| | | NT | NT | Active | AAGACTTTCTA | GACUUUCUAUCA | CAAAUUCUGAUAG | 191/192/193 |
| | | | | | TCAGAATTTGC | GAAUUUG | AAAGUC | |
| | | | | | G | | | |
| ise2c.pk0 | translation | NT | NT | No | AAACTTAATCA | ACUUAAUCAUGG | UCGUCGUCCAUGA | 194/195/196 |
| 05.b9 | initiation | | | | TGGACGACGAC | ACGACGA | UUAAGU | |
| | factor | | | | A | | | |
| | | NT | NT | Active | AAAGAAGAAG | AGAAGAAGAAGA | CCCUUCUUCUUCU | 197/198/199 |
| | | | | | AAGAAGAAGG | AGAAGGG | UCUUCU | |
| | | | | | GAG | | | |
| | | NT | NT | Active | AAGATCAAGAG | GAUCAAGAGAAU | CCUCGACAUUCUC | 200/201/202 |
| | | | | | AATGTCGAGGA | GUCGAGG | UUGAUC | |
| | | | | | T | | | |
| ise2c.pk0 | SAR1 | 90, 80 | No | Active | AAAATCGTCGG | AAUCGUCGGUUU | GUCGCUAAAACCG | 203/204/205 |
| 02.m10 | | | | | TTTTAGCGACG | UAGCGAC | ACGAUU | |
| | | | | | T | | | |
| | | NT | NT | Active | AACTGTCAATA | CUGUCAAUAGGC | GCAUACUGCCUAU | 206/207/208 |
| | | | | | GGCAGTATGCG | AGUAUGC | UGACAG | |
| | | | | | T | | | |
| | | NT | NT | Active | AACCTGTACCA | CCUGUACCAACA | AGUGGUCUGUUGG | 209/210/211 |
| | | | | | ACAGACCACTG | GACCACU | UACAGG | |
| | | | | | G | | | |
| ise2c.pk0 | Elonga- | NT | NT | Active | AACCAAAAATG | CCAAAAAUGGGC | UUUCCUUGCCCAU | 212/213/214 |
| 01.c14 | tion | | | | GGCAAGGAAAA | AAGGAAA | UUUUGG | |
| | factor | | | | G | | | |
| | | NT | NT | Active | AACGTGGTATC | CGUGGUAUCACC | UAUCGAUGGUGAU | 215/216/217 |
| | | | | | ACCATCGATAT | AUCGAUA | ACCACG | |
| | | | | | T | | | |
| | | NT | NT | Active | AACAAAATGGA | CAAAAUGGACUC | CUCAGUGGAGUCC | 218/219/220 |
| | | | | | CTCCACTGAGC | CACUGAG | AUUUUG | |
| | | | | | C | | | |
| ise2c.pk0 | Elonga- | NT | NT | Active | AATCCGTGACT | UCCGUGACUAAC | AUUUUUGGUUAGU | 221/222/223 |
| 01.d16 | tion | | | | AACCAAAAATG | CAAAAAU | CACGGA | |
| | actor | | | | G | | | |
| | | NT | NT | Active | AACATTGTCGT | CAUUGUCGUCAU | GUGUCCAAUGACG | 224/225/226 |
| | | | | | CATTGGACACG | UGGACAC | ACAAUG | |
| | | | | | T | | | |
| ise2c.pk0 | phospho- | 75, 75 | No | No | AATTTGTGAGA | UUUGUGAGACUG | CGGCCACCAGUCU | 227/228/229 |
| 05.h3 | oligo- | | | | CTGGTGGCCGA | GUGGCCG | CACAAA | |
| | saccharide . . . | | | | A | | | |
| | | NT | NT | No | AATCTGATTGT | UCUGAUUGUAUU | GGGGGCGAAUACA | 230/231/232 |
| | | | | | ATTCGCCCCCT | CGCCCCC | AUCAGA | |
| | | | | | C | | | |
| | | NT | NT | No | AACACTCTAGT | CACUCUAGUUCU | AAUAGGCAGAACU | 233/234/235 |
| | | | | | TCTGCCTATTCT | GCCUAUU | AGAGUG | |
| ise2c.pk0 | myosin | NT | NT | No | AACACACATCA | CACACAUCACAA | UCCGCCAUUGUGA | 236/237/238 |
| 01.d21 | | | | | CAATGGCGGAT | UGGCGGA | UGUGUG | |
| | | | | | A | | | |
| | | NT | NT | No | AAGGATGGCAT | GGAUGGCAUCAU | CUUGCCGAUGAUG | 239/240/241 |
| | | | | | CATCGGCAAGA | CGGCAAG | CCAUCC | |
| | | | | | A | | | |
| | | NT | NT | No | AAAGGCTTCAT | AGGCUUCAUCGA | CGCGGUGUCGAUG | 242/243/244 |
| | | | | | CGACACCGCGA | CACCGCG | AAGCCU | |
| | | | | | A | | | |
| ise2c.pk0 | myosin | NT | NT | No | AAACTCCAATT | ACUCCAAUUAUA | AGUAGGUUAUAAU | 245/246/247 |
| 01.j9 | | | | | ATAACCTACTA | ACCUACU | UGGAGU | |
| | | | | | C | | | |
| | | NT | NT | Active | AAGTACAAGGA | GUACAAGGAUCU | GCCGAUCAGAUCC | 248/249/250 |
| | | | | | TCTGATCGGCA | GAUCGGC | UUGUAC | |
| | | | | | A | | | |
| | | 40 | No | NO | AAGACTTTCTT | GACUUUCUUCAU | GGGCCACAUGAAG | 251/252/253 |
| | | | | | CATGTGGCCCA | GUGGCCC | AAAGUC | |
| | | | | | T | | | |
| ise2c.pk0 | myosin | NT | NT | No | AAACAAAGTAT | ACAAAGUAUCGC | GGUGUAGGCGAUA | 254/255/256 |
| 02.f12 | | | | | CGCCTACACCG | CUACACC | CUUUGU | |
| | | | | | C | | | |
| | | NT | NT | No | AATAGCGTCGA | UAGCGUCGAUCU | UCGUUGAAGAUCG | 257/258/259 |
| | | | | | TCTTCAACGAC | UCAACGA | ACGCUA | |
| | | | | | T | | | |
| ise2c.pk0 | potassium | NT | NT | No | AACTCATAGAG | CUCAUAGAGCUU | ACACAUCAAGCUC | 260/261/262 |
| 01.b14 | channel | | | | CTTGATGTGTG | GAUGUGU | UAUGAG | |
| | amino | | | | G | | | |
| | acid | | | | | | | |
| | trans- | | | | | | | |
| | porter | | | | | | | |
| | | NT | NT | Active | AAGATGTGGAT | GAUGUGGAUGAC | CAGUGACGUCAUC | 263/264/265 |
| | | | | | GACGTCACTGG | GUCACUG | CACAUC | |
| | | | | | T | | | |
| | | NT | NT | Active | AACCTTCCTGA | CCUUCCUGAUUC | CAGAAGAGAAUCA | 266/267/268 |
| | | | | | TTCTCTTCTGTG | UCUUCUG | GGAAGG | |
| ise2c.pk0 | potassium | NT | NT | Active | AACAGTGCTTG | CAGUGCUUGUGA | UCACUUAUCACAA | 269/270/271 |
| 03.f2 | inwardly | | | | TGATAAGTGAA | UAAGUGA | GCACUG | |
| | rectifier . . . | | | | C | | | |
| | | NT | NT | Active | AAGTTAATGGT | GUUAAUGGUGAC | GAGGGCAGUCACC | 272/273/274 |
| | | | | | GACTGCCCTCG | UGCCCUC | AUUAAC | |
| | | | | | A | | | |
| | | 90, 60 | Active | Active | AATAAAGCGAT | UAAAGCGAUGAC | CUAUGGGGUCAUC | 275/276/277 |
| | | | | | GACCCCATAGG | CCCAUAG | GCUUUA | |
| | | | | | A | | | |
| ise2c.pk0 | amino | NT | NT | Active | AAACGGTACTG | ACGGUACUGCAG | CUUUUUGCUGCAG | 278/279/280 |
| 05.l20 | acid | | | | CAGCAAAAAGA | CAAAAAG | UACCGU | |
| | trans- | | | | C | | | |
| | porter | | | | | | | |
| | | NT | NT | Active | AAGCTGCATAC | GCUGCAUACUUC | GAGCCAAGAAGUA | 281/282/283 |
| | | | | | TTCTTGGCTCTC | UUGGCUC | UGCAGC | |
| | | NT | NT | Active | AAATGTTTACA | AUGUUUACAGAG | AUCGCGUCUCUGU | 284/285/286 |
| | | | | | GAGACGCGATG | ACGCGAU | AAACAU | |
| | | | | | A | | | |
| ise2c.pk0 | tubulin | NT | NT | Active | AACGTCGATCT | CGUCGAUCUUAC | GAACUCGGUAAGA | 287/288/289 |
| 01.d1 | | | | | TACCGAGTTCC | CGAGUUC | UCGACG | |
| | | | | | A | | | |
| ise2c.pk0 | tubulin | NT | NT | Active | AATTCAAAATG | UUCAAAAUGCGU | UGCACUCACGCAU | 290/291/292 |
| 01.k6 | | | | | CGTGAGTGCAT | GAGUGCA | UUUGAA | |
| | | | | | C | | | |
| | | NT | NT | Active | AAATCGTAGAC | AUCGUAGACCUA | CGAGGACUAGGUC | 293/294/295 |
| | | | | | CTAGTCCTCGA | GUCCUCG | UACGAU | |
| | | | | | C | | | |
| ise2c.pk0 | tubulin | NT | NT | Active | AAACTCAATTC | ACUCAAUUCAAA | CACGCAUUUUGAA | 296/297/298 |
| 01.12 | | | | | AAAATGCGTGA | AUGCGUG | UUGAGU | |
| | | | | | G | | | |
| | | 90 | Active | No | AACTTATCACT | CUUAUCACUGGU | CUUCCUUACCAGU | 299/300/301 |
| | | | | | GGTAAGGAAGA | AAGGAAG | GAUAAG | |
| | | | | | T | | | |
| ise2c.pk0 | ubiquitin | NT | NT | Active | AAGAGTTACGA | GAGUUACGAACC | UGGUGACGGUUCG | 302/303/304 |
| 02.b4 | protein | | | | ACCGTCACCAT | GUCACCA | UAACUC | |
| | ligase | | | | A | | | |
| | | NT | NT | Active | AAACTTAGTCC | ACUUAGUCCGGA | UUCAUUAUCCGGA | 305/306/307 |
| | | | | | GGATAATGAAC | UAAUGAA | CUAAGU | |
| | | | | | C | | | |
| | | NT | NT | Active | AAGGCGATGTA | GGCGAUGUACGA | CAGGUUCUCGUAC | 308/309/310 |
| | | | | | CGAGAACCTGT | GAACCUG | AUCGCC | |
| | | | | | T | | | |
| ise2c.pk0 | small | NT | NT | Active | AACGACAAGAT | CGACAAGAUGCU | CUCCUUCAGCAUC | 311/312/313 |
| 01.j16 | nuclear | | | | GCTGAAGGAGA | GAAGGAG | UUGUCG | |
| | ribonu- | | | | C | | | |
| | cleo- | | | | | | | |
| | protein | | | | | | | |
| ise2c.pk0 | small | NT | NT | Active | AAGATAAAGGT | GAUAAAGGUCGC | UCCACACGCGACC | 314/315/316 |
| 06.h23 | nuclear | | | | CGCGTGTGGAC | GUGUGGA | UUUAUC | |
| | ribonu- | | | | C | | | |
| | cleo- | | | | | | | |
| | protein | | | | | | | |
| | | NT | NT | Active | AATGTCAAGAC | UGUCAAGACUGA | GUUUGGAUCAGUC | 317/318/319 |
| | | | | | TGATCCAAACA | UCCAAAC | UUGACA | |
| | | | | | C | | | |
| | | NT | NT | Active | AACATTCGAGT | CAUUCGAGUCUG | ACCUGUUCAGACU | 320/321/322 |
| | | | | | CTGAACAGGTG | AACAGGU | CGAAUG | |
| | | | | | G | | | |

### Example 2

### Transformation of Maize

Immature maize embryos from greenhouse donor plants are bombarded with a plasmid containing a silencing element of the invention operably linked to a maize Ubil-5UTR-Ubil intron and the selectable marker gene. PAT (Wohlleben et al. (1988) Gene 70:25-37), which confers resistance to the herbicide Bialaphos. In specific embodiments, the construct will have 2 identical 2-300 Bp segments of the target gene in opposite orientations with an “intron” segment between them such that a hairpin loop forms. Such a construct can be linked to a dMMB promoter. The plasmid further comprises a suppressor enhancer element comprising the target pest sequence or a fragment or variant thereof. Alternatively, the selectable marker gene is provided on a separate plasmid. Transformation is performed as follows. Media recipes follow below.

### Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising the silencing element of interest operably linked to a maize Ubil-5UTR-Ubil intron is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 12.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in a particle gun. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for the appropriate marker.

For example, a FAW feeding assay could be performed. In such assays, leaf discs from the transgenic plant are excised using a 1 cm cork borer or leaf punch. Six leaf discs are prepared for each plant. The leaves are placed in a 24 well microtiter plate on top of 500 μl of 0.8% agar. Each leaf disc is infested with 2 neonate fall armyworms (or any pest of interest) and the plate is then sealed with mylar. A small ventilation hole is made for each well and the plates are then stored in a 28° C. growth chamber. The assay is scored for mortality, stunting and leaf consumption at 96 hours.

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Geirite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/1 myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 3

### Agrobacterium-Mediated Transformation of Maize

For Agrobacterium-mediated transformation of maize with a silencing element and a suppressor enhancer element of the invention (for example, those described in example 2), the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the polynucleotide comprising the silencing element and the suppressor enhancer element to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

### Example 4

### Soybean Embryo Transformation

### Culture Conditions

Soybean embryogenic suspension cultures (cv. Jack) are maintained in 35 ml liquid medium SB196 (see recipes below) on rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 ml of fresh liquid SB196 (the preferred subculture interval is every 7 days).

Soybean embryogenic suspension cultures are transformed with the plasmids and DNA fragments described in the following examples by the method of particle gun bombardment (Klein et al. (1987) Nature, 327:70).

### Soybean Embryogenic Suspension Culture Initiation

Soybean cultures are initiated twice each month with 5-7 days between each initiation.

Pods with immature seeds from available soybean plants 45-55 days after planting are picked, removed from their shells and placed into a sterilized magenta box. The soybean seeds are sterilized by shaking them for 15 minutes in a 5% Clorox solution with 1 drop of ivory soap (95 ml of autoclaved distilled water plus 5 ml Clorox and 1 drop of soap). Mix well. Seeds are rinsed using 2 1-liter bottles of sterile distilled water and those less than 4 mm are placed on individual microscope slides. The small end of the seed are cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB196 liquid media for 7 days.

### Preparation of DNA for Bombardment

Either an intact plasmid or a DNA plasmid fragment containing the silencing element and suppressor element (such as those described in example 2) and the selectable marker gene are used for bombardment. Plasmid DNA for bombardment are routinely prepared and purified using the method described in the Promega™ Protocols and Applications Guide, Second Edition (page 106). Fragments of the plasmids carrying the silencing element of interest are obtained by gel isolation of double digested plasmids. In each case, 100 ug of plasmid DNA is digested in 0.5 ml of the specific enzyme mix that is appropriate for the plasmid of interest. The resulting DNA fragments are separated by gel electrophoresis on 1% SeaPlaque GTG agarose (BioWhitaker Molecular Applications) and the DNA fragments containing silencing element of interest are cut from the agarose gel. DNA is purified from the agarose using the GELase digesting enzyme following the manufacturer's protocol.

A 50 μl aliquot of sterile distilled water containing 3 mg of gold particles (3 mg gold) is added to 5 μl of a 1 μg/μl DNA solution (either intact plasmid or DNA fragment prepared as described above), 50 μl 2.5M CaCl₂ and 20 μl of 0.1 M spermidine. The mixture is shaken 3 min on level 3 of a vortex shaker and spun for 10 sec in a bench microfuge. After a wash with 400 μl 100% ethanol the pellet is suspended by sonication in 40 μl of 100% ethanol. Five μl of DNA suspension is dispensed to each flying disk of the Biolistic PDS1000/HE instrument disk. Each 5 μl aliquot contains approximately 0.375 mg gold per bombardment (i.e. per disk).

Tissue Preparation and Bombardment with DNA

Approximately 150-200 mg of 7 day old embryonic suspension cultures are placed in an empty, sterile 60×15 mm petri dish and the dish covered with plastic mesh. Tissue is bombarded 1 or 2 shots per plate with membrane rupture pressure set at 1100 PSI and the chamber evacuated to a vacuum of 27-28 inches of mercury. Tissue is placed approximately 3.5 inches from the retaining/stopping screen.

### Selection of Transformed Embryos

Transformed embryos were selected either using hygromycin (when the hygromycin phosphotransferase, HPT, gene was used as the selectable marker) or chlorsulfuron (when the acetolactate synthase, ALS, gene was used as the selectable marker).

### Hygromycin (HPT) Selection

Following bombardment, the tissue is placed into fresh SB 196 media and cultured as described above. Six days post-bombardment, the SB 196 is exchanged with fresh SB 196 containing a selection agent of 30 mg/L hygromycin. The selection media is refreshed weekly. Four to six weeks post selection, green, transformed tissue may be observed growing from untransformed, necrotic embryogenic clusters. Isolated, green tissue is removed and inoculated into multiwell plates to generate new, clonally propagated, transformed embryogenic suspension cultures.

### Chlorsulfuron (ALS) Selection

Following bombardment, the tissue is divided between 2 flasks with fresh SB196 media and cultured as described above. Six to seven days post-bombardment, the SB196 is exchanged with fresh SB196 containing selection agent of 100 ng/ml Chlorsulfuron. The selection media is refreshed weekly. Four to six weeks post selection, green, transformed tissue may be observed growing from untransformed, necrotic embryogenic clusters. Isolated, green tissue is removed and inoculated into multiwell plates containing SB196 to generate new, clonally propagated, transformed embryogenic suspension cultures.

Regeneration of Soybean Somatic Embryos into Plants

In order to obtain whole plants from embryogenic suspension cultures, the tissue must be regenerated.

### Embryo Maturation

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 uE/m2s. After this time embryo clusters are removed to a solid agar media, SB166, for 1-2 weeks. Clusters are then subcultured to medium SB103 for 3 weeks. During this period, individual embryos can be removed from the clusters and screened for the appropriate marker or the ability of the plant, when ingested by the pest, to control the pest.

### Embryo Desiccation and Germination

Matured individual embryos are desiccated by placing them into an empty, small petri dish (35×10 mm) for approximately 4-7 days. The plates are sealed with fiber tape (creating a small humidity chamber). Desiccated embryos are planted into SB71-4 medium where they were left to germinate under the same culture conditions described above. Germinated plantlets are removed from germination medium and rinsed thoroughly with water and then planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot. After 10 to 16 weeks, mature seeds are harvested, chipped and analyzed for proteins

### Media Recipes

SB 196 - FN Lite liquid proliferation medium (per liter) -

| | | |
|---|---|---|
| MS FeEDTA - 100x Stock 1 | 10 | ml |
| MS Sulfate - 100x Stock 2 | 10 | ml |
| FN Lite Halides - 100x Stock 3 | 10 | ml |
| FN Lite P, B, Mo - 100x Stock 4 | 10 | ml |
| B5 vitamins (1 ml/L) | 1.0 | ml |
| 2,4-D (10 mg/L final concentration) | 1.0 | ml |
| KNO3 | 2.83 | gm |
| (NH4)2 SO 4 | 0.463 | gm |
| Asparagine | 1.0 | gm |
| Sucrose (1%) | 10 | gm |
| pH 5.8 | | |

### FN Lite Stock Solutions

| Stock # | | 1000 ml | | 500 ml | |
|---|---|---|---|---|---|
| 1 | MS Fe EDTA 100x Stock | | | | |
| | Na₂ EDTA * | 3.724 | g | 1.862 | g |
| | FeSO₄—7H₂O | 2.784 | g | 1.392 | g |
| 2 | MS Sulfate 100x stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—5H₂O | 0.0025 | g | 0.00125 | g |
| 3 | FN Lite Halides 100x Stock | | | | |
| | CaCl₂—2H₂O | 30.0 | g | 15.0 | g |
| | KI | 0.083 | g | 0.0715 | g |
| | CoCl₂—6H₂O | 0.0025 | g | 0.00125 | g |
| 4 | FN Lite P, B, Mo 100x Stock | | | | |
| | KH₂PO₄ | 18.5 | g | 9.25 | g |
| | H₃BO₃ | 0.62 | g | 0.31 | g |
| | Na₂MoO₄—2H₂O | 0.025 | g | 0.0125 | g |

* Add first, dissolve in dark bottle while stirring

SB1 solid medium (per liter) comprises: 1 pkg. MS salts (Gibco/BRL—Cat# 11117-066); 1 ml B5 vitamins 1000× stock; 31.5 g sucrose; 2 ml 2,4-D (20 mg/L final concentration); pH 5.7; and, 8 g TC agar.

SB 166 solid medium (per liter) comprises: 1 pkg. MS salts (Gibco/BRL—Cat# 11117-066); 1 ml B5 vitamins 1000× stock; 60 g maltose; 750 mg MgCl2 hexahydrate; 5 g activated charcoal; pH 5.7; and, 2 g gelrite.

SB 103 solid medium (per liter) comprises: 1 pkg. MS salts (Gibco/BRL—Cat# 11117-066); 1 ml B5 vitamins 1000× stock; 60 g maltose; 750 mg MgCl2 hexahydrate; pH 5.7; and, 2 g gelrite.

SB 71-4 solid medium (per liter) comprises: 1 bottle Gamborg's B5 salts w/ sucrose (Gibco/BRL—Cat# 21153-036); pH 5.7; and, 5 g TC agar.

2,4-D stock is obtained premade from Phytotech cat# D 295—concentration is 1 mg/ml.

B5 Vitamins Stock (per 100 ml) which is stored in aliquots at −20 C comprises: 10 g myo-inositol; 100 mg nicotinic acid; 100 mg pyridoxine HCl; and, 1 g thiamine. If the solution does not dissolve quickly enough, apply a low level of heat via the hot stir plate. Chlorsulfuron Stock comprises 1 mg/ml in 0.01 N Ammonium Hydroxide

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A plant cell comprising
a) a first heterologous polynucleotide comprising a silencing element for a target pest sequence operably linked to a promoter active in the plant cell; and,
b) a second heterologous polynucleotide comprising a suppressor enhancer element comprising the target pest sequence or a fragment or variant thereof operably linked to a promoter active in the plant cell.

2. The plant cell of claim 1, wherein the combined expression of said silencing element and the suppressor enhancer element increases the concentration in said plant cell of an inhibitory RNA specific for the pest target sequence.

3. The plant cell of claim 1, wherein the first and the second polynucleotide are stably incorporated into the genome of said cell.

4. The plant cell of claim 1, wherein said pest is an insect pest.

5. The plant cell of claim 4, wherein said insect pest is selected from the group consisting of:
a) a member of the Lygus genus;
b) an aphid;
c) a member of the family Aphididae; and
d) a member of the Lepidoptera order.

6. The plant cell of claim 5, wherein said member of the Lepidoptera order comprises Spodoptera frugiperda.

7. The plant cell of claim 1, wherein said silencing element encodes a hairpin RNA.

8. The plant cell of claim 7, wherein said siliencing element comprises, in the following order, a first segment, a second segment, and a third segment, wherein
a) said first segment comprises at least about 18 nucleotides having at least 90% sequence complementarity to the target pest sequence;
b) said second sequence comprises a loop of sufficient length to allow the silencing element to be transcribed as a hairpin RNA; and,
c) said third segment comprises at least about 18 nucleotides having at least 85% complementarity to the first segment.

9. The plant cell of claim 1, wherein said plant cell is from a dicot.

10. The plant cell of claim 9, wherein said dicot is soybean, Brassica, sunflower, cotton, or alfalfa.

11. The plant cell of claim 1, wherein said plant cell is from a monocot.

12. The plant cell of claim 11, wherein said monocot is maize, wheat, rice, barley, sorghum, or rye.

13. A plant or plant part comprising the cell of claim 1.

14. The plant or plant part of claim 13, wherein the combined expression of said silencing element and the second polynucleotide increases the concentration of an inhibitory RNA specific for the pest target sequence in the phloem of said plant or plant part.

15. A transgenic seed from the plant of claim 13.

16. A method for increasing the concentration of inhibitory RNA specific for a target pest sequence comprising introducing into a plant cell, a first polynucleotide comprising a siliencing element for the pest target sequence and a second polynucleotide comprising a suppressor enhancer element comprising the target pest sequence or a variant or fragment thereof,
wherein the combined expression of said siliencing element and the second polynucleotide increases the concentration in said plant cell of an inhibitory RNA specific for the pest target sequence in said plant cell.

17. The method of claim 16, wherein said first polynucleotide and said second polynucleotide are stably incorporated into the genome of said plant cell.

18. The method of claim 16, wherein the combined expression of said siliencing element and the suppression enhancer element increases the concentration of an inhibitory RNA specific for the pest target sequence in the phloem of a plant comprising the plant cell.

19. The method of claim 16, wherein said pest in an insect pest.

20. The method of claim 19, wherein said pest is selected from the group consisting of:
a) a member of the Lygus genus;
b) an aphid;
c) a member of the family Aphididae; and,
c) a member of the Lepidoptera order.

21. The method of claim 20, wherein said member of the Lepidoptera order comprises Spodoptera frugiperda.

22. The method of claim 16, wherein said silencing element encodes a hairpin RNA.

23. The method of claim 22, wherein said polynucleotide comprising the siliencing element comprises, in the following order, a first segment, a second segment, and a third segment, wherein
a) said first segment comprises at least about 18 nucleotides having at least 90% sequence complementarity to the target polynucleotide;
b) said second segment comprises a loop of sufficient length to allow the silencing element to be transcribed as a hairpin RNA; and,
c) said third segment comprises at least about 18 nucleotides having at least 85% sequence complementarity to the first segment.

24. The method of claim 16, wherein said plant cell is from a dicot.

25. The method of claim 24, wherein said dicot is soybean, Brassica, sunflower, cotton, or alfalfa.

26. The method of claim 16, wherein said plant cell is from a monocot.

27. The method of claim 26, wherein said monocot is maize, wheat, rice, barley, sorghum, or rye.

28. A method for controlling a pest comprising
feeding to the pest a plant cell comprising a first polynucleotide comprising a siliencing element for a pest target sequence and a second polynucleotide comprising a suppressor enhancer element comprising the target pest sequence or a fragment or variant thereof, wherein the level of expression of the pest target sequence or the polypeptide encode thereby is reduced.

29. The method of claim 28, wherein the combined expression of said siliencing element and the second polynucleotide increases the concentration in said plant cell of an inhibitory RNA specific for the pest target sequence.

30. The method of claim 28, wherein the combined expression of said siliencing element and the second polynucleotide increases the concentration of an inhibitory RNA specific for the pest target sequence in the phloem of a plant comprising said plant cell.

31. The method of claim 28, wherein said first polynucleotide and said second polynucleotide are stably incorporated into the genome of said plant cell.

32. The method of claim 28, wherein said pest in an insect pest.

33. The method of claim 32, wherein said pest is selected from the group consisting of:
a) a member of the Lygus genus;
b) an aphid;
c) a member of the family Aphididae; and,
d) a member of the Lepidoptera order.

34. The method of claim 33, wherein said member of the Lepidoptera order comprises Spodoptera frugiperda.

35. The method of claim 28, wherein said siliencing element comprises a hairpin RNA.

36. The method of claim 35, wherein said polynucleotide comprising the siliencing element comprises, in the following order, a first segment, a second segment, and a third segment, wherein
a) said first segment comprises at least about 18 nucleotides having at least 90% sequence complementarity to the target polynucleotide;
b) said second segment comprises a loop of sufficient length to allow the silencing element to be transcribed as a hairpin RNA; and,
c) said third segment comprises at least about 18 nucleotides having at least 85% sequence complementarity to the first segment.

37. The method of claim 28, wherein said plant cell is from a dicot.

38. The method of claim 37, wherein said dicot is soybean, Brassica, sunflower, cotton, or alfalfa.

39. The method of claim 28, wherein said plant cell is from a monocot.

40. The method of claim 39, wherein said monocot is maize, wheat, rice, barley, sorghum, or rye.


SEQUENCE LISTING (322 sequences)

SEQ ID NO: 1 - DNA, 540 bp, Spodoptera frugiperda
  misc_feature 417, 423, 500, 524, 540: n = A,T,C or G
caagcatccaacatggtatccgacttcaggaagaagaagctcctccacgtgttcaagtccttcttcgacacggacggcagcggcaacatcgagaaggatgacttcctgatggccatcgaaaggataaccaagaccagaggctggaaagctggagacgacaaatacaaatttgtcgaggagaccctattgaagatctgggacggcatccagaaggtcgctgacgagaacaaggacggacaggtcagccaggacgagtggatcgctatgtgggacaagtactccaagaacccgtccgaggcgttcgagtggcagaccctgtactgcaagttcgcgttcactcttgaagacgccagcgacgatggatccatcgacagcgaggagttctcctctgtgtacgcctccttcggcctggacaangacgangctgtggctgccttcaagaaagatggctaacggtaagtccgaagtgtcctgggcttgagttccacgacctgtggaangagtacttctcatccggaagactngaacgctgccggcaan

SEQ ID NO: 2 - DNA, 505 bp, Spodoptera frugiperda
  misc_feature 358, 410, 442, 482: n = A,T,C or G
ccaacatggtatccgacttcaggaagaagaagctcctccacgtgttcaagtccttcttcgacacggacggcagcggcaacatcgagaaggatgatttcctgatggccatcgaaaggataaccaagaccagaggctggaaagctggagacaacaaatacaaatttgtcgaggaaaccctattgaagatctgggacggcatccagaaggtcgctgacgagaacaaggacggacaggtcagccaggacgagtggatcgctatgtgggacaagtactccaagaacccatccgaggcgttcgagtggcagaccctgtactgcaagttcgcgttcactcttgaagacgccagcgacgacggatncatcgacagcgaagagttctcctctgtgtacgcctccttcgggctggacaanggacgaggcggtggctgccttcaagaagatgntaacggtaagtccgaatgtcctggggctgagtttcaaganctgttggaaggatacttctcaac

SEQ ID NO: 3 - DNA, 410 bp, Spodoptera frugiperda
  misc_feature 271, 346: n = A,T,C or G
caacatggtatccgacttcaggaagaataagctcctccacgtgttcaagtccttcttcgacacggacggcagcggcaacatcgagaaggatgacttcctgatggccatcgaaaggataaccaagaccagaggctggaaagctggagacgacaaatacaaatttgtcgaggagaccctattgaagatctgggacggcatccagaaggtcgctgacgagaacaaggacggacaggtcagccaggacgagtggatcgctatgtgggacaagtantccaagaacccgtccgaggcgttcgagtggcagaccctgtactgcaagttcgcgttcactcttgaagacgccagngacgatggatccatcgacagcgaggagttctcctctgtgtacgcctccttcggcctggacaagg

SEQ ID NO: 4 - DNA, 445 bp, Spodoptera frugiperda
  misc_feature 66, 72, 264, 329, 358, 404, 406, 413, 427, 443: n = A,T,C or G
gagagagagagagagagagaactagtctcgagtttttttttttttttttttttttttttttttttnggaaantactattttattgtaccaactgccccttaacctcatctatgagtcacccataaatgttattttggtaaaatgtttgacacacttcacactaatatttataaatgtgaaagtttgtttgtttgaatgtttgtatatttgtctgtcaatcacgctgaaaccactgtatagaatttgacctaatttggtatacanacagggtatgagctgacttgggtgataggatactttttatcccacaggaacgcgggtaaagtccntgggcagaagctagtatgtaataattatntccctctacctaccctatatgggggtggaccgtcatgttctttacncnacaaccngtttgtccacctcncctttaaagttttgtnag

SEQ ID NO: 5 - DNA, 672 bp, Spodoptera frugiperda
  misc_feature 652, 653, 654, 655, 656, 657, 658, 670, 671, 672: n = A,T,C or G
gcacgagggccgtgtcgacttcgcaccagtcccctatttatttaccttgacaaaaatatggcgcgcctattgtttattgcgcctatcctggcgttggctataatgccagtatacttcttattcctaaagggaccacccccactacccgaactagatatgaacgagtggtggggcccagagaagctaaaagcaaaacctgacactagtataaaaccctttaaaattgcttttggagacactgttgtaaaagacttaaaagaccgtctcaaacgttctcggtctttcactgctccgctggagggtgtggcattccagtacggcttcaacactgctcagctggatggttggctgaagtactgggctaatgagtataagttcaaggagagagagaccttcctcaaccagtaccctcagtacaaaaccaatatccagggtcttgacatccacttcatcagggttacaccgaaggtaccggcaggagtggaggtggtacccatgctactcctccacggctggccaggctctgtcagggagttctacgaggctattcctctcatcacagcagtcagcaaggaccgtgacttcgctgtggaagtcatcgttccaagtctacctggctatggattctctgatgccgcagttcgtcccggcnnnnnnnccccacaaatgnnn

SEQ ID NO: 6 - DNA, 693 bp, Spodoptera frugiperda
gcacgaggcttggacgtgatgttacctgggaattcaaccccttgaatgttaaggtcggctcccacatcaccggaggagacttgtacggtatcgtacacgagaacacattggttaagcacaagatgttgatcccacccaaggccaagggtaccgtcacctacgtcgcgccctccggcaactacaaagtcactgacgtagtgttggagacggagttcgacggcgagaaggagaagtacaccatgttgcaagtatggccggtgcgccagccgcgccccgtcactgagaagctgtccgccaaccaccccctgctcaccggacagagagtgctcgactctctcttcccttgtgtccagggtggtaccacggccatccccggcgccttcggttgtggcaagactgtcgtctcacaggctctgtccaagtactccaactctgacgtcatcatctacgtcggatgcggtgaacgtggtaacgagatgtctgaggtactgcgtgacttccccgagctgacggtggagatcgagggcatgaccgagtccatcatgaagcgtaccgcgctcgtcgccaacacctccaacatgcctgtagccgcccgagaggcttccatctacaccggtatcaccctctccgagtacttccgtgacatgggttacaacgtgtccatgatggctgactccacctctcgttgggccg

SEQ ID NO: 7 - DNA, 300 bp, Spodoptera frugiperda
  misc_feature 39, 40, 41, 162, 164, 166, 167, 168, 169, 242, 268, 269,270, 293, 294, 295, 299, 300: n = A,T,C or G
gcacgaggcagatagtcatcactgtttttgggacctgtnnntactccctcaataaacctacaaaatggccgaaaacccaatctacggacccttctttggagttatgggggcggcgtctgctatcatctttagcgcgctgggagctgcctatggaactgctangncnnnnaccggtatcgccgccatgtcggtgatgcggcccgagctcatcatgaagtccaacaactacaccctttacaagnggttcatccaccttggcgctggtctnnncgtaagtttctccggtctagcgnnnggcnn

SEQ ID NO: 8 - DNA, 688 bp, Spodoptera frugiperda
  misc_feature 318, 319, 320, 321, 658, 659, 660, 661, 662, 686, 687,688: n = A,T,C or G
gcacgaggctcacaggctctgtccaagtactccaactctgacgtcatcatctacgtcggatgcggtgaacgtggtaacgagatgtctgaggtactgcgtgacttccccgagctgacggtggagatcgagggcatgaccgagtccatcatgaagcgtaccgcgctcgtcgccaacacctccaacatgcctgtagccgcccgagaggcttccatctacaccggtatcaccctctccgagtacttccgtgacatgggttacaacgtgtccatgatggctgactccacctctcgttgggccgaggctcttcgtgagatctcnnnncgtctggctgagatgcctgccgactcgggttaccccgcctacctgggagcccgtctggcctcgttctacgagcgtgccggacgtgtgaagtgcttgggtaaccccgacagggagggctccgtgtccatcgtgggcgccgtgtcgccgcccggaggtgacttctccgaccccgtgacggccgccacgctgggtatcgtgcaggtgttctgggggttggacaagaagctcgcgcagcgcaagcacttccccgccatcaactggctcatctcctacagcaagtacatgcgagcgctggacgacttctatgagaagaactaccccgagttcgtgcccctcnnnnncaagggtcaaggagatcctgcagnnn

SEQ ID NO: 9 - DNA, 685 bp, Spodoptera frugiperda
  misc_feature 674, 675: n = A,T,C or G
gcacgaggtatctaaaacagtgcgtcgtaatatattcaagatgtctcgtcttaggttttgttttttattagcagtactatgcagttgtttgcagaatggttacggttttacaacagaaaagccagttacccagcatgtagatcctaaaccagaagttcctgaaacgttgcctgaaacaacacgagtgcctgcgccgagctcgtcgacggcagcgccgaccacaccagctccgacaccggcaccaacgccagcacccacaccagctcctacaccagctcctactccagctcctacccctgcgcctactcctgcgcctactcctgcgcctactcctgcgcctacccccgcacctacaccagcgcccactcctgctcccaccccagctcccctccccgcccccgaccaaggcacatggtccttcactgatgaaaaggccaatcagacatgcattgtggcccaattcgcagcccaactgaatgtcacatacaccaagttagtggagaatgcaacgtctctatcgtacgtgaggctcaacgtgcccgcgaacgcgtcggtcctcaacggcagctgttcggaccccgaccaatggatccagatcacctggaagaccaacgacgacagcgagacgaacaacaccatgaccctcgtgtacaacaagaatgccaccaccaagnnctacggcctg

SEQ ID NO: 10 - DNA, 612 bp, Spodoptera frugiperda
  misc_feature 558, 560, 573, 574, 575, 589, 590, 591, 592, 593, 595,596, 597, 599, 604, 605, 612: n = A,T,C or G
gcacgagggcggtttgaagtgatctagttcgtcagaaaaaacacagaccacgttcacaatgaaatcgatggtggtgttattcgctgtgtgcgccgtggcgtgcggctccctggtgccgctggcgcagcctcctcatcaccccgccgtcgtgctggacccgcacggccgcccgctcgacaccgccgaggtgatcaacgcccgcgccctccacctgcaggctaaggccctggatggacactacgctcccctcgcgcacgctgccgtcgtgcctgttgcccactccgtggtagccgcccccgctgtggtcgccgctcccgccgccgtgtcccaccagtcccgtgtggatgtgcgcaccagccccgccatcgtgagccacgccgtcgctgctcccgtagtagcccacggtgtctactccgctcccctgctggcccactccgctctcggctacgccggtcacggacactacctgaagaagcgctccctgggacacctcgcctacgccgctcccgtcgtcgcccacgtagctccctccgcggtgtcgcaccagtcccgcgtggncntcgtctccagccnnnctgtcgtgtctcannnnntnnntnccgtnntgtcccn

SEQ ID NO: 11 - DNA, 550 bp, Spodoptera frugiperda
  misc_feature 460, 461, 485, 486, 487, 488, 489, 499, 501, 527, 528,529, 535, 536, 537, 538, 539, 540, 541, 542: n = A,T,C or G
gcacgaggggacgttgaacgaaagaaaatgctacgcgttacgattttagccgcagtggtggtgttcgcctcaggcgcgccccagaacaacttcatcttcaagaatgacatcactcctgaggaagcccagcagtacctcaaacaactgccgttcacctcaccccagctctctggacgcaccgctgtactgcctctggttcgctacgacgaccccaggtttcgttcagctgaagctggcccaacccttggacactactggaagaatggacaggagatccagaacacagaggactacttagaagaggtctacaacgcggctcaataccacggccaggacggtcttggcaactacgcctacggttatgagacccctgaatcttccaaggttgagaaccgtgaaggttccggagtcgtccaaggatcctatgtgtaccaggttcccggaatgaaggatctcgtcnnggtccgttactgggctgacagccnnnnnttccaccagnangacaatcttcccaaggttgaactgannnccgctnnnnnnnncccgctct

SEQ ID NO: 12 - DNA, 687 bp, Spodoptera frugiperda
  misc_feature 286, 287, 288, 632, 633, 634, 635, 636: n = A,T,C or G
gcacgaggtatcactcctgaccgtatctaaaactcggcacacaacacaatggctgacatcgaagatacacatttcgagaccggggactccggtgcctccgccaccttccctatgcaatgctcggccctgcgcaagaacggtttcgtcatgcttaagggtcgcccctgcaaaatcgtcgagatgtccacttccaaaaccggaaagcacggccacgctaaagttcacttggttggaatcgatatttttaacggcaagaaatacgaagatatctgcccttccacccacnnncatggacgtgccccacgtgaagcgtgaggactaccagctcaccgatatctctgacgacggctaccttaccctcatggctgacaacggcgatctccgcgaggacctcaagatcccagacggtgacctcggcacccagttgcgttctgacttcgatagcggcaaagagctgttgtgcactgtgctgaagtcttgcggtgaggagtgtgtaatcgcagtcaaggcaaacacagctctcgacaaataaaccaactcagcatttatagggatatacatacatataatttttttacaatcaacagctcttacataaatgtaaaacataatactatgtataatttaacatnnnnnattatggtgtgacgcggtgctggcttgtcgccgtccactccacccccgaag

SEQ ID NO: 13 - DNA, 514 bp, Spodoptera frugiperda
  misc_feature 486, 487, 488, 510, 511, 512, 513, 514: n = A,T,C or G
gcacgaggcgcgattgtaacatgtcgtattcaccagaaagaagatcagaagattggccggaagattccaaaaatggcccgtctaaggatcaaggcaactatgatgggcctccaggaatggaaccccaaggggcacttgatacaaactggcaccaggtcgtggaaagctttgacgacatgaatctgaaggaagaattgttgagaggaatttatgcttacggttttgaaaagccgtctgctatccaacaacgcgctattatgccttgcattcaaggccgtgatgtcatagctcaagcccagtctggtactgggaagactgctaccttctctatttcaattcttcagcaaatcgataccagtattcgtgaatgccaagcactgattttggcccctactagagagctggctcagcagatccaaaaggtggtgattgctcttggggatcacttgaatgctaaatgccatgcttgcatcggcggcactaatnnngcgcgaagatgttcgtcagctnnnnn

SEQ ID NO: 14 - DNA, 636 bp, Spodoptera frugiperda
  misc_feature 635, 636: n = A,T,C or G
gcacgagggtcgtattcaccagaaagaagatcagaagattggccggaagattccaaaaatggcccgtctaaggatcaaggcaactatgatgggcctccaggaatggaaccccaaggggcacttgatacaaactggcaccaggtcgtggaaagcttcgacgacatgaatctgaaggaagaattgttgagaggaatttatgcttacggttttgaaaagccgtctgctatccaacaacgcgctattatgccttgcattcaaggccgtgatgtcatagctcaagcccagtctggtactgggaagactgctaccttctctatttcaattcttcagcaaatcgataccagtattcgtgaatgccaagcactgattttggcccctactagagagctggctcagcagatccaaaaggtggtgattgctcttggggatcacttgaatgctaaatgccatgcttgcatcggcggcactaatgtgcgcgaagatgttcgtcagctggagagtggtgtgcatgtggtggtgggtacacctggtcgcgtgtacgacatgataactcgtcgtgctctccgtgctaacactatcaagctgtttgtacttgatgaagctgatgaaatgctgtcaagaggatttaaagatcnn

SEQ ID NO: 15 - DNA, 592 bp, Spodoptera frugiperda
  misc_feature 578, 579, 580, 583, 584, 585: n = A,T,C or G
gcacgagggccatcctgtcacacatctaccaccacgccctgcacgataactggttccaagctcgagacttgctcttgatgtcacacttgcaagagactgttcaacattcagacccgagcactcagattttgtacaatcgtactatggccaatctaggtttgtgcgcttttcgaaggggcaatgttaaagaagcccatggctgcctagctgaactgatgatgactggcaaacccaaggaactgttagctcaaggtctgctacctcagcgtcaacacgagcgttcaaaggaacaggaaaagatagagaagcaacgccaaatgccgttccacatgcacatcaacttggaactgcttgaatgtgtgtatttagtgtctgccatgctgattgaaattccatacatggccgcccacgaattcgatgctcgccggcgcatgattagtaagactttctatcagaatttgcgcgcaagtgagcgtcaggctttggtaggcccgcccgaatccatgcgtgagcatgctgtggctgccgccagggcgatgcgccgcggagactggcgtgcttgcctcaattttattgtnnntgnnnaatgaat

SEQ ID NO: 16 - DNA, 609 bp, Spodoptera frugiperda
  misc_feature 448, 449, 450, 452, 595, 596, 597, 598, 599: n = A,T,C or G
gcacgaggctgatagccacctgccaaattatcttgaaatataaccattcactaaaatatttaacgtaatttagtggttaattctaaacttaatcatggacgacgacatggtatttgatccatctttaaagaaaaagaagaagaagaagaccggtttcgacttagatgccgctctcgcaggcgaacaaggtgagagcacgagcgtggaggcgcccgctgggtcgggtgacgtcgacttgcctgaggatgataacctcgatttggataattttggaaagaaaaagaagaagaagaagaagggagtcttcaacatggaagaacttgaaagtacgttaccggaaacacctccggccgaagagccggaacagcaggaggacgaagttattgacgatttagatctagatattgacttctctaaaacgaaaaagaagaagaagaagaaaaacatnnnangagctcgtccttgaagatgacaccaagggagaagatcaagagaatgtcgaggatgttagtggtgatttatggagcggcacagaccgtgactacacgtacgacgagctactagagcgagtgttcgacatcatgcgagaaaagannnnnagcatggttt

SEQ ID NO: 17 - DNA, 639 bp, Spodoptera frugiperda
  misc_feature 511, 512, 614, 616, 618, 628, 631, 635, 636, 637, 638: n = A,T,C or G
gcacgaggcagattcatatttccatcgcttattcgttgctgagaaaaatcgtcggttttagcgacgtaacatattgctaataagtgtgaaatattgtgataaacttccttttagcattagttaatctagttcaattttaaataattcaaaatgtttatcttggattggttcactggtgttctcggattccttggtctgtggaagaaatcaggcaagctactgttcctgggactggacaatgctggcaagaccacactcctgcacatgctgaaggatgacagattggcgcagcatgtacccacattgcatcccacgtcggaggaactgtcaataggcagtatgcgtttcacgacgttcgacttgggcgggcatcagcaggcgcggcgcgtgtggcgcgactacttcccggcggtggacgccatcgtgttcctggtggacgcgtgcgaccgcccgcgcctgcccgagtccaaggccgagctggactcgctgctcactgacgagacgctcagcnnactgccccgtgctcatcctcggcaacaagatcgacaagcccggcgcagctagtgaggacgagctccgtcagttcttcaacctgtaccaacagaccactgganangncaaagtatcnagntcannnnt

SEQ ID NO: 18 - DNA, 639 bp, Spodoptera frugiperda; identical to SEQ ID NO: 17
  misc_feature 511, 512, 614, 616, 618, 628, 631, 635, 636, 637, 638: n = A,T,C or G

SEQ ID NO: 19 - DNA, 574 bp, Spodoptera frugiperda
  misc_feature 72, 73, 74, 190, 191, 192, 568, 569, 570, 571, 572, 573,574: n = A,T,C or G
gcacgagggtctatctcggatattacacgtggattgtaatccgtgactaaccaaaaatgggcaaggaaaagnnncacattaacattgtcgtcattggacacgtcgactccggcaagtccaccaccaccggtcacttgatctacaaatgcggtggtatcgacaaacgtaccatcgagaagttcgagaaggnnncccaggaaatggggtaagggttccttcaaatacgcctgggtattggacaaactgaaggctgagcgtgaacgtggtatcaccatcgatattgctctgtggaagttcgaaaccgctaaatactatgtcaccatcattgacgctcccggacacagagatttcatcaagaacatgatcactggaacttcccaggctgattgcgccgtactcattgtcgccgctggtaccggtgagttcgaggctggtatctcgaagaacggacagacccgtgagcacgctctgctcgctttcacactcggtgtcaagcagctgattgtgggcgtcaacaaaatggactccactgagcccccatacagcgaatcccgtttcgaggaaatcnnnnnnn

SEQ ID NO: 20 - DNA, 169 bp, Spodoptera frugiperda
  misc_feature 64, 153, 154, 155, 165, 166, 167, 168, 169: n = A,T,C or G
gcacgaggcggatattacacgtggattgtaatccgtgactaaccaaaaatgggcaaggaaaagnttcacattaacattgtcgtcattggacacgtcgactccggcaagtccaccaccaccggtcacttgatctacaaatgcggtggtatcgannnacgtaccatnnnnn

SEQ ID NO: 21 - DNA, 169 bp, Spodoptera frugiperda; identical to SEQ ID NO: 20
  misc_feature 64, 153, 154, 155, 165, 166, 167, 168, 169: n = A,T,C or G

SEQ ID NO: 22 - DNA, 690 bp, Spodoptera frugiperda
  misc_feature 505, 506, 507, 508, 510, 511, 512, 526, 527, 528, 529,530, 531, 592, 593, 594, 595, 678, 679, 680, 682, 683, 684: n = A,T,C or G
gcacgaggctctagtcccgtcaccgtcgccagtagggggcgccacaagaacagaaagagaattatttcaaactccaattataacctactagataactccaaaagttctgtcagttctaactttaatttaacggggacgtcagagtttatggataggaccgataagataatatcggacgcgactgagctacaagcaatgcagaactttatcatggagaagatttacgaaatggaacctaatgagaagaagaagcaatctgaggtcgacagggtattcaaacacgcattattagaattcaaagacaatttagtagcgacgtacagcatagtggagacgcggggctctgcgctgaagtacaaggatctgatcggcaacttcctgcacgtcatggagacggtttgtgccagggaggggtccacgctctccatcaccatgggggtcaacgcctttaggggtttcatggacgagtttatgagccaacatgacactgataaagctaggacgnnnngnnnaaggataaaaagannnnnntggacgatccaatacaatacaaaggccatacgttcatactgtccatgatcaacataccaannnnagtgtgagatctgcaagactttcttcatgtggcccatagagcggtcactcatatgccagacgtgtaaacttgcctcgcataannntnnnacacta

SEQ ID NO: 23 - DNA, 711 bp, Spodoptera frugiperda
  misc_feature 612, 613, 614, 663, 664, 665, 667, 668, 701, 706, 707,708, 709, 710, 711: n = A,T,C or G
tgactccacagtgggacaaactcatagagcttgatgtgtggtacgctgctgtgacccaagtgttcttctctctgtctgtgtgcaccggtgccatcattatgttctcgtcctacaatggattcagacaaaatgtttacagagacgcgatgattgtcactactttggacaccttcaccagtttgttatccggtttcacgatcttcggtatcctgggtaacttggcgtacgagttggacaaagatgtggatgacgtcactggttctgcaggaactggacttgccttcatttcataccctgacgcgatctccaaaactttccagccacagttgttcgcagtgctgttcttcttgatgatgacggtactaggtatcggatcagcagttgctttactttccaccatcaacaccgtgatgatggacgcgttccctcgcatcaagaccatctacatgtccgccttctgctgcactattggatttgccatcggtctcatttacgtcacacctggtggccaatatattctcgagctggtggattacttcggtggaaccttcctgattctcttctgtgctatcgctgaaattattggtgtattctggatttacggcttggagnnntatgcctggatattgagtacatgttgggagttaaacttcttctactggnnntnntgttggggcgttattatgcctgccatgatgatnaccgnnnnnn

SEQ ID NO: 24 - DNA, 625 bp, Spodoptera frugiperda
  misc_feature 540, 541, 542, 543, 614, 615: n = A,T,C or G
gcacgagggtaaacagattttaacactacattaatttgttctagagttaaatgtattaattccgacttaaaaacagtgcttgtgataagtgaacacaaattattgagcaatgactgactttataaaacaatatttcaaggaacaatatgaaataaatgaaaaaatgctttcgaaaattgacgcggatctgcgaacctgcggagcacacttagtagcagtgaagttaatggtgactgccctcgagttgaaaatgacttcgatgaagacaatgtatcaggatctaatggaactcagagaaataatcgttcttttaaatccacacttgaagaaaccgagataataatacaatacagtaaggttaacgaatactatcttttaatttccttaaattatgttcataaaaatgattaagttgtttagctgaacacagtggtgtactgacaggataggtttcattaaactttgcataatcgatcagaaaaccgtgcttttcttttttgtactcgaccatttcaataaagcgatgaccccataggattnnnntgggtggtgtagctcgacttcgcttggacaggctgaccagttgattctatagtgccttcaaacactacgannatttccatat

SEQ ID NO: 25 - DNA, 472 bp, Spodoptera frugiperda
  misc_feature 454, 455, 456, 463, 464, 465: n = A,T,C or G
gcacgaggattttcttaaaacggtactgcagcaaaaagacggcattgaaggtggactcggtctgcctatctggtacctggtggtttgtctgttcgggtcatggtttatcatcttcgtgattgtgtcccgaggtgtaaagagttccggtaaagctgcatacttcttggctctcttcccctacgttgtgatgctcattttgcttataacgacctctattctgcccggagccggcaccggcattcttttcttcctgactccacagtgggacaaactcatagagcttgatgtgtggtacgctgccgtgacccaagtgttcttctctctgtctgtgtgcaccggtgccatcattatgttctcgtcctacaatggattcagacaaaatgtttacagagacgcgatgattgtcactactttggacaccttcaccagtttgttatccggtttcacgatcttnnntatcctnnntaacttg

SEQ ID NO: 26 - DNA, 676 bp, Spodoptera frugiperda
  misc_feature 563, 564, 565, 566, 648, 649, 650, 655, 656, 666, 667,669, 670, 671, 674, 675, 676: n = A,T,C or G
gcacgaggccggtcttcagggcttccttatcttccactccttcggtggaggtactggatctggtttcacttccctcctgatggagcgactctccgtggactacggcaagaagtccaagctggagttcgccatctacccggcgcctcaggtgtccaccgctgtcgtggagccctacaactccatcctcaccacccacaccacccttgagcactccgactgcgccttcatggtcgacaacgaggccatctacgacatctgccgccgcaacctcgacatcgagcgccccacgtacaccaacctgaaccgtctcatcgggcagatcgtgtcctccatcacggcctccctgcgcttcgacggcgccctcaacgtcgatcttaccgagttccagaccaacttggtgccctacccccgtatccacttccctctggtcacatacgccccggtcatctctgccgagaaggcgtaccacgagcagctgtcggtggctgaaatcaccaacgcatgcttcgagcccgccaaccagatggtcaagtgcgaccctcgtcacggcaagtacatggctnnnntgcatgttgtaccgtggtgacgtcgtccccaaggacgtgaacgccgccatcgccaccatcaagaccaagcgtaccatccagnnncgtcnnttggtgtccnncnnngtnnn

SEQ ID NO: 27 - DNA, 650 bp, Spodoptera frugiperda
  misc_feature 522, 523, 524, 525, 526, 533, 534, 535, 536, 537, 600,601, 621, 622, 623, 625, 628, 629, 633, 634, 635, 636, 637, 640,641, 642, 643, 644, 645: n = A,T,C or G
gcacgaggattcgtttggcaagcctcttaaccggtcgcgctgaacgacgactgatatttaattaatttatattctacgttaagttcaacaaaactcaattcaaaatgcgtgagtgcatctcagtacacgttggacaagccggagtccagatcggtaatgcctgctgggaattatattgccttgagcatggaatccagcctgacggccagatgcccacagacaagaccgtgggcggtggtgatgactccttcaacaccttcttcagcgagaccggtgccggcaagcacgtccccagggctgtgtttgttgacttggaacccacagtagttgatgaggtccgcactggcacatacagacagttgtttcatccagaacaacttatcactggtaaggaagatgcggccaacaactacgcccgtggtcactacaccatcggcaaggaaatcgtagacctagtcctcgaccgcatccgtaagctcgccgaccagtgcaccggtctccagggcttccttatcttccacnnnnntcggtgnnnnnactgggatctggtttcacttccctcctgatggagcgactctccgtggactacggcaagaagtnnaagctggagttcgccatctnnncngcnnctcnnnnntcnnnnnnctgtc

SEQ ID NO: 28 - DNA, 621 bp, Spodoptera frugiperda
  misc_feature 521, 522, 523, 525, 532, 534, 535, 536, 596, 597, 618,619, 620, 621: n = A,T,C or G
ttcggcacgaggggcaagcctcttaaccggtcgcgctgaacgacgactgatatttaattaatttatattctacgttaagttcaacaaaactcaattcaaaatgcgtgagtgcatctcagtacacgttggacaagccggagtccagatcggtaatgcctgctgggaattatattgccttgagcatggaatccagcctgatggccagatgcccacagacaagaccgtgggcggtggtgatgactccttcaacaccttcttcagcgagaccggtgccggcaagcacgtccccagggctgtgtttgttgacttggaacccacagtagttgatgaggtccgcactggcacatacagacagttgtttcatccagaacaacttatcactggtaaggaagatgcggccaacaactacgcccgtggtcactacaccatcggcaaggaaatcgtagacctagtcctcgaccgcatccgtaagctcgccgaccagtgcaccggtctccagggcttccttatcttccactccnnncngtggagntnnntggatctggtttcacttccctcctgatggagcgactctccgtggactacggcaagaagtnnaagctggagttcgccatctannnn

SEQ ID NO: 29 - DNA, 602 bp, Spodoptera frugiperda
gcacgaggatcaaagagttacgaaccgtcaccatactgaaggagataccattcgtcgtgccattctcaacacgcgtccttatattccaaggacttttagcgagagagaagcacgaccactggtacgaaatgacgaacttcaacgaggggccctcgatcaacatcagtgttcgaaggacgcatttatatgaagatgcatttgataaacttagtccggataatgaacctgatttgaagttgaaacttcgcgtgcaactgatcaaccaggccggtgcggaggaagctggtgtcgacggcggtggactattccgagagtttctttctgagctcttaaaatctgcatttgatccgaacaggggtctgttccggctgacaatagacaacatgttgtatccgaaccccgccgtacatctactgtacgatgacttccccatgcactactacttcgtcggcaggatgctgggaaaggcgatgtacgagaacctgttggtggagctgccgctggcggagttcttcctgggcaagctgtgcggctgcggggaggccgacgtgcacgcgctggcctcgctcgaccccgcgctgcaccgcgggttgttactactc

SEQ ID NO: 30 - DNA, 639 bp, Spodoptera frugiperda
  misc_feature 602, 603, 625, 626, 627, 628, 629: n = A,T,C or G
gcacgaggtgtacagccacttgctgtaccacaggcaacaggcggaggagcctagagcgagggatgagcaggagatcgacggtggcgcgacagttggctgcttgacatcagggcagagtggagggggctgcggctgccggctgagcacgtgtgttggagtaggaccacacggcatcgtcgtctacagaccagcctgcaacggagaacaagaaattggagtcgagaaacaaagtatcgcctacaccgccatccaccgcgcacagcctttccgacgcaacttccaaatgtcgttcgtcacggacgagggcaatgaagccactcttcacatcaagatggccacgtcggggcaggcagccgccctctaccgcgccgtcacggagaaacatgcgttctactactgcgagactgtcaggactgctgttactgaacagttcaccagagatctgaagggcacaatagcgtcgatcttcaacgactctacgactctagggcggcgttacgtgtttgacatccgacgcacgtgccgcgaggtatacgaccgcgcacgccgcgccgtgcatgcgcagcaacactccgccgcgccgcacgactcgcgcgcacgannccgcaccgcctcgggctcggannnnnggagtcccgg

SEQ ID NO: 31 - DNA, 299 bp, Spodoptera frugiperda
  misc_feature 222, 223: n = A,T,C or G
gcacgagggccggccgccgtgttcgtgccgtcccgccgggccgcgcgcctactggccgccgacctgctggcgctggccgcggcgcacgcgcagcccgccgccttcctgcgcgcgcgccccgacgtgctgcagcccttcctcaagaggatcaacgacaagatgctgaaggagacggtggctgcgggcgtggcgtacctgcacgagggcgtggacccggcggannggcgcctggtgcaacaactgctggagtcgggcgcgctggcgctctgcgtcgtggccgccgagctggcctggggact

SEQ ID NO: 32 - DNA, 624 bp, Spodoptera frugiperda
  misc_feature 562, 563, 564, 620, 621, 622, 624: n = A,T,C or G
gcacgaggcgaagataaaggtcgcgtgtggaccttaggtttaagtttattattaaataatttagcctaaacataagtcatggccaataacgacaactttgcacaagatgttactgataatcaactaaatggaaatgccgaaaatggtggtggcgatacgcaagaacataatagtgccgaagcccctgggcgtgatgatgacagaaaactttttgtcggaggcctgagctgggaaaccacagacaaggagttacgtgaccacttcagtgcatatggtgagattgagagcatcaatgtcaagactgatccaaacactggcagatcaagaggatttgcctttattgtgttcaaggcaccagattcaatagacaaagtgatggctgctggagagcacactattaacaacaaaaaagttgatccgaaaaaagcaaaggctagacatggaaagatctttgttggtggtcttagcagtgaaatatcagatgatgagatcaaaaacttcttcagtaattttggaacaataattgaagtcgagatgccctttgacaaaaccaagaatcagnnnaagggattctgctttataacattcgagtctgaacaggtggtcaatgagctgctgannncn

SEQ ID NO: 33 - DNA, 644 bp, Spodoptera frugiperda
  misc_feature 607, 621, 622, 623, 634, 635, 636, 637, 638, 639, 640: n = A,T,C or G
gcacgaggcgcgtgtggaccttaggtttaagtttattattaaataatttagcctaaacataagtcatggccaataacgacaactttgcacaagatgttactgataatcaactaaatggaaatgccgaaaatggtggtggcgatacgcaagaacataatagtgccgaagcccctgggcgtgatgatgacagaaaactttttgtcggaggcctgagctgggaaaccacagacaaggagttacgtgaccacttcagtgcatatggtgagattgagagcatcaatgtcaagactgatccaaacactggcagatcaagaggatttgcctttattgtgttcaaggcaccagattcaatagacaaagtgatggctgctggagagcacactattaacaacaaaaaagttgatccgaaaaaagcaaaggctagacatggaaagatctttgttggtggtcttagcagtgaaatatcagatgatgagatcaaaaacttcttcagtaattttggaacaataattgaagtcgagatgccctttgacaaaactaagaatcagaggaagggattctgctttataacattcgagtctgaacaggtggtcaatgagctgctgangactcctaagcagnnnattggtggcannnnnnncgac

SEQ ID NO: 34 - DNA, 580 bp, Spodoptera frugiperda
  misc_feature 569, 570, 571: n = A,T,C or G
gcacgaggatgaagttggctctgacactcttggctctggcggcggtggccaccgctaaaaacatcaacgtcgaggatgccatcgacctagaggacatcaccgcctacggatacttggctaagatcggtaaacctcttgccgacgaaatccgcaaagctgaggaggcagagagcgcatccagaattgttggtggtcaggcctccagcctcggacagttcccctaccaggctggtcttctcgctgacttctccgctggccaaggtgtgtgtggtggttccttggtgcgtgccaaccgtgttcttactgctgctcactgctggttcgatggccagaaccaggcctggagattcaccgttgttcttggctccatccgtttgttctccggtggtaccagagttcaaacctccaacgttgttatgcatggaagctggaaccccagtaacatccgtaatgacgtcgccatgatcaggctgaactccaacgttggtctttcaaacaccattgcactcatcgctctgcccagcggtagccagctcaacgaaaacttcgccggtgaaaacgccgtcgcnnnctggattcg

SEQ ID NO: 35 - DNA, 676 bp, Spodoptera frugiperda
  misc_feature 648, 649, 650, 651, 652, 653, 660, 661, 671, 672, 673,674, 675, 676: n = A,T,C or G
gcacgaggatcaaaatgaaactgttcctcgcagtcgtgtgcttggccgttgccgcatccgcggtggagattggagttccgtctcaggaaaacccagtctttggctaccatcaaaacttcggtattgccgaagctgccaggatcaagaaggctgaggaagaaaccagccctagcgcccagaggatcgtcggaggatctgtcactgacatttccaacgtcccttaccaggctggtctcgtgatccaagttttggtcatcttccaatccgtgtgcggtggttccatcatctcccacaaccgcatcgtgaccgctgctcactgcaactgggacggttctatcaccgctaactctttcaccgtcgtacttggctccaacttcctcttctccggcggtaaccgcatcaccaccagagatgttgtcatgcaccccaactggaccccaaccaccgctgccaacgacattgctgtcctccgcattagctccgttactttcaccaacgtgatccagcccatcgctctgcccagcggcaacgagctcaacaacgacttcgtcaactggaacgctatcgcttccggatacggtcttaccgctgatggtgctaacatcggtactacccaacgtgtcagctccgtggtactccccgtgatcnnnnnncgccagnncgctaccgtnnnnnn

SEQ ID NO: 36 - DNA, 611 bp, Spodoptera frugiperda
  misc_feature 590, 591, 592, 604, 605, 611: n = A,T,C or G
gcacgaggaatcttagttacattggagtgacttttatttatcaataacatttttatttgaagactcagtacgtattatcgcgtagttcaacagagttgctagtgtagttttctgaaagttgccatcttgcttttgcaacttttaaatataaaagtcttattagatcgtttttactaccgataaatttactaaaaatataaaagtgcaatttacaattactctgttagtgtcagtttgtgtgaatttgtcgtagttataaaaggacactgtattgattttgtcaatcagtttgacgcatgcgctcattgggtgccgtaaaaaagggttggccaacattccgaacagtgtcgttccggtcgccgttgtcgtggtgtcggtgaagttagtggtggaatttttacgtgtataacatcaaaaaatggcgtctggtgtgacagtttcggacgcgtgcaaaacgacgtacgaggagattaagaaagacaagaagcaccgctacgtggtgttctacatcagggatgagaaacaaattgacgtagagaccgtcggcgaacgtaacgcggaatacgatcagttccttgaggatctgcagnnnggtggcaccggnnagtgcn

SEQ ID NO: 37 - DNA, 674 bp, Spodoptera frugiperda
  misc_feature 497, 498, 499, 672, 673, 674: n = A,T,C or G
gcacgaggctgatatctaatcttagttacattggattgacttttatttatcaataacatttttatttgaagactcagtacgtattatcgcgtagttcaacggagttgctagtgtagttttctgaaagttgccatcttgcttttgcaacttttaaatataaaagtcttattagatcgtttttactaccgataaatttatcaaaaatataaaagtgcaatttacaattactctgttagtgtcagtttgtgtgaatttgtcttagttataaaaggacactgtattgattttgtcaatcagtttgacgcatgcgctcattgggtgccgtaaaaaagggttggccaacattccgaacagtgtcgttccggtcgccgttgtcgtggtgtcggtgaagttagtggtggaatttttacgtgtataacatcaaaaaatggcgtctggtgtgacagtttcggacgcgtgcaaaacgacgtacgaggagattaagaaagacaagaagnnnccgctacgtggtgttctacatcagggatgagaaacaaattgacgtagagaccgtcggcgaacgtaacgcggaatacgatcagttccttgaggatctgcagaagggtggcaccggagagtgcagatatggcctcttcgacttcgagtacacgcaccagtgccaaggcacgtcgnnn

SEQ ID NO: 38 - DNA, 684 bp, Spodoptera frugiperda
  misc_feature 648, 649, 650, 652, 653, 654, 657, 659, 660, 661, 662,667, 680, 681, 682, 683, 684: n = A,T,C or G
gcacgaggcctcgtgccgcgcgaatagacagttttgtgtgcacaatgttgatcctttggctaaatatcatcgcaataatttgtgtcataccctacgcaaatggagaaggaagggttgcaatagcgcatttacaatcgctaaagtcagtgactggtcaaattcaatttacggagacggcaaaagggcttcatgtcgaaggagttatatttggtttaccacccggtgcctacgggtttcacgttcacgaattaggagatgttgcacctggttgcgaccaggcgggccggcacttcaaccctgagggatccacccacggtggcaggaactccaccgtacgccatgtcggtgacctcggaaatgtagtgttcgttagcgagcgagccgcttatgctacagtagactttgtagatagtctattggcacttcaaggacgtaatagtatattggggcgctctttggtcttgcatgaacaaacggatgacctaggtttgggaggaaacgcgacgtctttgactacaggtaactcggggccccggatagcatgtggtgctattggaatcaaatcaccttatgacccttggaatgctgctagctctatgtctccgtcgatgctactatttatcacatctttaactttatttactttannntnnnaantnnnngtatnagtatttaatttnnnnn

SEQ ID NO: 39 - DNA, 402 bp, Spodoptera frugiperda
  misc_feature 321, 322, 323, 324, 325, 354, 355, 366, 398, 399, 400,402: n = A,T,C or G
gcacgaggcttccacatacgcgaatagacagttttgtgtgcacaatgttggtcctttggctaaatatcatcgcaataatttgtgtcataccctacgcaaatggagaaggaagggttgcaatagcgcatttacaatcgctaaagtcagtgactggtcaaattcaatttacggagacggcaaaagggcttcatgtcgaaggagttatatttggtttaccacccggtgcctacgggtttcatgttcacgaattaggagatgttgcacctggttgcgaccaggcgggccggcacttcaaccctgagggatccaaccacggtggcnnnnnctccaccgtgcgccatgtcggtgacctcnnaaatgtagtgnttgttagcgagcgagccgcttatgctacagtnnncn

SEQ ID NO: 40 - DNA, 627 bp, Spodoptera frugiperda
  misc_feature 393, 394, 395, 396, 397, 435, 436, 437, 439, 619, 620,621, 622: n = A,T,C or G
gcacgagggtcgagagatacggtgcgcacatagcaacaatatcaaagtacaaaggtcagtaactatgagtggtaaattgttaaaaactctaatccttggggcacctgcttcaggcaaggggactatatcgtctcggatagtgaagaaatatgctgtggcacacgtgtccagtggggacaagctgagggaccacattgagaaacaaactgacctaggtaaagaagtcaaaaagtacttgaatgaagggaaacttgtacctgatgatgtcatgataaagtttatgatcacagaattaaaaaaagttgaagataaaccatggctactggatggattcccgaggactgtgggacaggctgatgctttgtggaaggtacaacctgttgatgtagtagnnnnnttagtagtgccttttgaggtaatcatagacagagtgannnancgctgggtgcacttgccttcgggccgagtgtataacattggcttcaacactcctaaagtggaaggtaaggatgatgagacaggtgaggacttggttcagagacctgacgacaagccagaggctgtgcgcaagcggctggagatctatgagagtgtgacgaggccagtcatagagttctannnngctaa

SEQ ID NO: 41 - DNA, 627 bp, Spodoptera frugiperda; identical to SEQ ID NO: 40
  misc_feature 393, 394, 395, 396, 397, 435, 436, 437, 439, 619, 620,621, 622: n = A,T,C or G

SEQ ID NO: 42 - DNA, 395 bp, Spodoptera frugiperda
  misc_feature 330, 331, 332, 333, 358, 359, 360, 361, 362, 363, 368,369, 370, 371, 372, 373, 374, 376, 377, 378, 380, 381, 382, 386,388, 389, 390, 391, 392, 393, 394, 395: n = A,T,C or G
tgctgctgctggaagctgggcccaaccctcccgaggagagcattataccaggcttaagacaaaccttgaaagaaacgccctacgactggaacttcaccaccattgacgacggggtcacgagccaggcgctggcgggccacgtgcagagacagccgcggggcaagatgctgggcggcagcggctcgctcaacgacatggtgtacgcgcggggccaccccgaggactactacgagtgggccgacatcgccggcgacgtctggaactggaccaacgtgctggactacttcaagcggacggagcacatgacggacgccaatatcgttcacaacnnnnagctcatgcagtaccacggcacggnnnnnnccatnnnnnnntnnngnnnccantnnnnnnnn

SEQ ID NO: 43 - DNA, 570 bp, Spodoptera frugiperda
  misc_feature 555, 556, 557, 561, 562, 563, 564, 565, 567, 568, 569,570: n = A,T,C or G
gcacgaggggaaaacatgggaaggaggtcgcatcaagatgttagtgctcgacttgaactgcccggtcgttggagacgactgcaaagacagccgcaagaagttgcttgtggactacttccatacaaacctgcatacccagaacttctacgcgttccgcttctttatctgcgaagtgttgaacttcatcaacgtcgtgggccagatcttcttcatggactttttcctggacggcgagttctccacgtacggcagtgacgtggtcagtttcaccgagatggagcccgaggagcgtgtggacccgatggctagagtgttcccgaaagtgaccaagtgcaccttccacaaatacggtccttcaggaaccgtgcagaagttcgacggtctgtgcgtgctgccattgaacatcgtcaatgaaaagatctacgtgttcctgtggttctggtttatgatcctgtcgatcctgagtggaatttcgctgatttaccgcatggccgtggtggctggaccgcgcgtgcgcctgtacctgctgcgtgcgcgcagccgcctggccccgcnnncgcnnnnngnnnn

SEQ ID NO: 44 - DNA, 648 bp, Spodoptera frugiperda
  misc_feature 639, 640, 641, 643: n = A,T,C or G
gcacgaggattttaatagctattatgactttacagactagacggatcaaggccatgcctctcgcttgcatactcaccatccgcacataccgtattgcggtatgtcaataagttgcaaataatgtctgttcagttttacaaggataagatcagcagtatttgcgaactgtacctactactaagctgataatgtaataattaaactttattattgaaatagatatgtataattgacatctttctcaaatgggtgtcaatactgccaactctattaccacaatttcttttcgtatttgcttttatactgagcctgatgacgtactgtactttttattagaatttaatttttcttatttttcttactacgtagtcattaaatctgagaaattaaaaattactaatttagaactcccaaattctgaatgaggttctaaaaagttgttaggaatactaaataccattttaccaacataaatctaatttcgttacttaaaatattaaatgtataatgaaatgtctatgataagtgtttactatctttatatcgacaaaatttattttccatgttttaaaatttatttttcagatgttttgacgtgataagtttgtattttatcaatatctgatagtcgagagttannnantattg

SEQ ID NO: 45 - DNA, 716 bp, Spodoptera frugiperda
  misc_feature 612, 613, 614, 703, 704, 705, 706: n = A,T,C or G
gcacgagggaggagaggtggtggctggcttccttgcaaacgaagcgtcgtaaattacatcttatttgtaaattttaataaaaatttgatcgttaaacgatcgaatcagtagtgatttaagtgctcaagcagtttcacatccaatcgacaatgagttcgagtgtatgctacaagtgtaaccggacagggcacttcgcccgcgagtgcacccagggtggtgttgccgctcgtgactctggtttcaaccgtcagcgcgaaaagtgcttcaagtgcaaccgcgctgggcacttcgctcgggattgcaaggaggaggccgaccgttgctacagatgtaacggcacgggacacatagcgcgtgagtgcgcgcaaagtccggacgagccgtcgtgttacacttgcaacaagaccgggcacatcgcacggaactgcccagagggcgggcgcgacagctccaaccagacctgctacaactgcaacaagtccggccacatctcacgcaactgccccgacggcaccaagacttgttacgtgtgcggaaagcccggacacatctcccgcgattgcgatgaggagcggaactaacacacgcctcttcgcgactgcctatatatannntaaactatgtatattatgatgccacgcacggacgataagcaaaggacgcgatacgcgacactagatcgtaagaccacacgactgtatgnnnntaatgcaacg

SEQ ID NO: 46 - DNA, 473 bp, Spodoptera frugiperda
  misc_feature 472: n = A,T,C or G
gcacgaggataataaacgttaatatttaacaagttgaaaagtttgtctttcaatttgtgattttgtaaagatcattctatggaatggacagtttgctatctgtgaaacatccattagctttgtgttgagagcagaggtcgcggcggcggggtgatgcggccatggcttcgcggcgcgtgacgcgcaagtgggaggtgttcgcgggacggaaccgattctggtgcgacggccgcctcatgacggcgccgcaccccggcgtgttcctgctcacgctcgcgctcatctgcggcacgtgcgccctgcacttcgccttcgactgccccttcctggccgtgcgcgtgtcgcccgccgtgcccgcggccggcgccgcgctgtgcgcgctgacgctggcggcgctgctgcgcacggcgctgtccgaccccggcatcatcccgcgcgccgccgcggccgaggcggcggcgctggaggcggng

SEQ ID NO: 47 - DNA, 574 bp, Spodoptera frugiperda; identical to SEQ ID NO: 19
  misc_feature 72, 73, 74, 190, 191, 192, 568, 569, 570, 571, 572, 573,574: n = A,T,C or G

SEQ ID NO: 48 - DNA, 169 bp, Spodoptera frugiperda; identical to SEQ ID NO: 20
  misc_feature 64, 153, 154, 155, 165, 166, 167, 168, 169: n = A,T,C or G

SEQ ID NO: 49 - DNA, 514 bp, Spodoptera frugiperda; identical to SEQ ID NO: 13
  misc_feature 486, 487, 488, 510, 511, 512, 513, 514: n = A,T,C or G

SEQ ID NO: 50 - DNA, 535 bp, Spodoptera frugiperda
  misc_feature 15, 16, 358, 375, 376, 377, 378, 379, 426, 427, 428,450, 451, 452, 454, 457, 509, 510, 511, 512, 513, 514, 515, 516,517, 518, 519, 520, 521, 524, 525, 531, 532, 533, 534, 535: n = A,T,C or G
cgcgcacgtcgctcnncaagcccgctgcagcgccggccaagcccgcccccgcggcggcgcgcgccaccagtgcgaccagccgcgcggcccccgcggcccggccggcccccaagtccgcagtaggcgcagcgcggcccgcagcacaaaagacagatgcggccgccaaacccgcggcgacccgggttgcggctccgcgtcccgcgctgtcggcgcccaggccccagcctaagccggcagacaagaagccagtaccgaatggtgacgtgaaagactccaagccagccgcgcggcccgcgccccggccggccgcggccgcgcgccccgcgccgcgccccactccccgcgcccccgccgcacnggtcgcacccactactnnnnngagtgcccccaagccggcgccgcgtgctcccctggacaagcagagcnnngacctcgctaacaaacgcatcnnngncanggcagcaccgcctaggactgctccccctaagacgacaacgacgacaacaggnnnnnnnnnnnnngtnncgaagnnnnn

SEQ ID NO: 51 - DNA, 586 bp, Spodoptera frugiperda
  misc_feature 457, 458, 459, 460, 461, 462, 489, 505, 506, 507, 556,557, 558, 559, 560, 561, 567, 568, 569, 570, 571, 573, 577, 578,579, 580, 581, 586: n = A,T,C or G
gcacgaggctataacaagcagcatataaaaatgaaattcttgctgtctttcgctgccgtcatcgccgtggccgccgctggcctggtgcccgttggacccgccggccctgcgcccgctcctgaggcccctgaggtcttcgagcccgtcgctattggacccgctgtcattgactccttcgagcccatcgccatcggacccgctatcatcgactccttcgagcccatcgccatcggacccgctattgttccatctcccgagcccgtcgccatcggacccgccatcattgagagcccagagcccgttgctgtcggacctgcatggattgacttccccctgcccgacggtggtgctgccgttgcccccgttgagccctctcccgtggctgttatccccggtcccgtgtccactgaggttgcttcaggcactcccctcgttcagatcatcctgaacatcaacnnnnnntctgctgacgttagccccgttgctgtnggccccgctgtcgagnnnacacccgtgcacgttgtggactctgcccctgaacccgtccacgttgtgnnnnnngccccnnnnncnatcnnnnngtcgn

SEQ ID NO: 52 - DNA, 623 bp, Spodoptera frugiperda
  misc_feature 563, 564, 565, 566, 567, 568, 622, 623: n = A,T,C or G
gcacgaggcttagagtaagcataggtgtatttatgtattgagtcggaagaagcaatggacgatccaaataggatgatggcgcatagcggcgggcttatggggccgcagggctacggcctgcctggcggcgagggaactccaaccgcaggcgaaggtgaagcccgcaagcaagatattggtgaaatattgcaacagatcatgaatattacagatcaaagtcttgatgaagcgcaagcgagaaaacatactctcaactgtcacagaatgaagcctgccctattttcagtgttgtgtgaaatcaaagagaaaacagtgctgtccctccgcaacacgcaagaggaggagcccccagatccccagctgatgcgcttggacaacatgctcatagccgagggggtcgctggccctgaaaagggtggtggtgcgggcgctgcagcttcggcatcagctgctgctggtgaatgggacaatgccatcgagcactctgactaccgtgcgaagttggcgcagatccgccagatctaccaccaggagctggacaagtatgagaatgcttgtaatgnnnnnnccacccacgtgatgaacttactccgcgagcagagccgcaccaggcctatcacann

SEQ ID NO: 53 - DNA, 535 bp, Spodoptera frugiperda; identical to SEQ ID NO: 50
  misc_feature 15, 16, 358, 375, 376, 377, 378, 379, 426, 427, 428,450, 451, 452, 454, 457, 509, 510, 511, 512, 513, 514, 515, 516,517, 518, 519, 520, 521, 524, 525, 531, 532, 533, 534, 535: n = A,T,C or G

SEQ ID NO: 54 - DNA, 624 bp, Spodoptera frugiperda
  misc_feature 506, 507, 508, 622, 623, 624: n = A,T,C or G
gcacgaggccaggtttgagaaaaacgcttaaactgccacaaaatcccgttctcgaagaagcacttttcacttattaataagtaacttgtgtaaaatgtggtttaaatgtgtattttactaaacctcaataaatatatttatatcaaaatattttttttctatactgtattatttattcctatagtacatattataatccgaacgctccgtgagtccgaacaggggtaattttttggtagttcggattatcgaggctctactgtatacctactttttgttaaaatattttagtcttatatacgacttcctaactaatccatatctcttagagctttcgaatatccatttgcctttttcttaaaaagattaataactatttatatatatcccaaatatataaaaacaaccactccaattattattattcaaatatgacaaactagatagaatgtcccaagaaatttgcaaaaaagtaatgttcaaattattaaccgaagaacgaattnnngagtgtataatattatacagacatttagaaatttttaataggctccaatcgcatgagaggtcgctttaaaattcggcattggtgtgtgcgttgcaatttaatctttaacacccnnn

SEQ ID NO: 55 - DNA, 678 bp, Spodoptera frugiperda
  misc_feature 627, 631, 632, 634, 669, 670, 671, 672, 673, 674, 675: n = A,T,C or G
gcacgaggggacgtgtttacaatttactttcgtgctcgtgtgattttaattaaaacagtgctaagtgctctaggacgctgaataactgatatttgttttaaaagttgatataaattaatcacaatgaatagagataaacgagaaccagagtatccaacggagttggagtctcaattcgtaatgcgtttacctgaggagcctgcaaaagttttgagagaagtgttgaaatccggagagaacctgaaaaacagactgacgatacaaatagaaaacgacatgcgcacgggcgaggtaaggtttgatcactggttgatgcacgccaagatcgtggatctaccaaccatcatagaatctctaaaaacgatcgacaacaagagtttctacaaaacagcagatatatgccaaatgatgatttgtaaagaagaacctgaccaaccatccacagaggaagagtcaccagctaaaaataagaaaaaagatccatacaaagttgacaaaaagttcctatggccacacggcatcacaccgcctacgaagaacgtacggaagcgtcgatttagaaaaacccttaaaaagaaatatgtagaagcaccagaaattgaaaaggaagtgaagaggctgctgagngcanncnatgaggctgttagtgttaactgggaggtcatcaannnnnnngat

SEQ ID NO: 56 - DNA, 683 bp, Spodoptera frugiperda
  misc_feature 562, 563, 564: n = A,T,C or G
gcacgagggtcgaatggaacatggcggtgctaggcaggatgtgcataagtttttgatttttgcatttttaacgagttgcttatatcagttagctttctaaataatttctgacttatttcgtgtgttataatatttgttatagtgtaaaagcttatccaccccaggaatttcctatctggacttacttagttctgcaatgaaaattattattcgttggtagtgtaaaaataattgtgacaaatatatcactttgcttcagtgtgccgtgttggtcatggctacgctcctccaagagaatggtataaaggagttaagcaaagttgtgcctaaccgtggtatatcctcacatagtgtaacaaatcatatggtgcctgatcatgaatattgcgaagctgggtcaactagcacgtcacagatgaagtgtaccgatacaagtgaggcgatggcgccacccgccgccattgaagaagaggaggatacaccagaaatagatataatgataaacaatgttgtgtgcagttttagtgttaagtgccacctgaaccttagacagatagcattnnntggtgtgaacgttgaatttcgccgcgagaacggcatggtaactatgaagttacggcgtccatacactactgcgtccatctggtcgtccggccgcgtgacgtgcactggtgcaaccagcg

SEQ ID NO: 57 - DNA, 658 bp, Spodoptera frugiperda
  misc_feature 621, 643, 644, 645, 646, 654, 655, 656, 657: n = A,T,C or G
gcacgagggaatattgcgaagctgggtcaactagcacgtcacagatgaagtgtaccgatacaagtgaggcgatggcgccacccgccgccattgaagaagaggaggatacaccagaaatagatataatgataaacaatgttgtgtgcagttttagtgttaagtgccacctgaaccttagacagatagcattaaatggtgtgaacgttgaatttcgccgcgagaacggcatggtaactatgaagttacggcgtccatacactactgcgtccatctggtcgtccggccgcgtgacgtgcactggtgcaaccagcgaggaccaggcgaaggttgccgcacgacggtatgcgcgcgcccttcagaagctcggcttccaagtgcgtttccgcaatttccgtgtagtcaatgtattaggcacctgtcggatgccgtttggtataaggatcatatctttttcgaaaaaatacaaggaagcagactatgaacctgagctccatcctggagtcacatataagttatacaatcctaaagccacactcaagatattctccactggtggtgtgactatcacagctcggagtgtgagtgacgttcagtcagccgtggaacgcatcttccctttgntgtacgagttccgcaagcctcnnnnaccggcannnna

SEQ ID NO: 58 - DNA, 652 bp, Spodoptera frugiperda
  misc_feature 640, 643, 644, 645, 646: n = A,T,C or G
gcacgagggtaccaaaagctcttttcattgcagctgaagggtcactgcaacttggccaatcagaattagcattgaaactattcaaagaactaaaacaagaaggaatggaaatcaggcaacatttctattggcctttgttagttcagaaggcaaaggaaaatgatgaggaaggcctcttgcaaattttaaaagaaatgagcagcaatgactttactgttactggagaagcgttaagagactatgttatcccttacttgataaaaaaagattctccacagaatgtcttacttaaacttcaaattgcaaatgtaccaacaatccatgctgcaagaaatctaatggttgatcttttggattctggagacataaaaggcgcagcggaaatagctctgcaatatagaccttggggcaactactctcttgttgccaggtccctcatcaatgcagtgaataagacaaaagatgtagaatcgtttgctaaaattcttcatgctataagcagtaaacctttgtcacagggtgaagaagatgttgctgccaacaatgaggaaggtcaaagtgatgaaaataatgatattcatgaagtcggccgtattgtgaggtcgtctgccaagagtttggctaaaccagacttaatagnaannnntttaga

SEQ ID NO: 59 - DNA, 23 bp, Spodoptera frugiperda
aacatggtatccgacttcaggaa

SEQ ID NO: 60 - RNA, 19 nt, Spodoptera frugiperda
caugguauccgacuucagg

SEQ ID NO: 61 - RNA, 19 nt, Spodoptera frugiperda
ccugaagucggauaccaug

SEQ ID NO: 62 - DNA, 23 bp, Spodoptera frugiperda
aaggtcgctgacgagaacaagga

SEQ ID NO: 63 - RNA, 19 nt, Spodoptera frugiperda
ggucgcugacgagaacaag

SEQ ID NO: 64 - RNA, 19 nt, Spodoptera frugiperda
cuuguucucgucagcgacc

SEQ ID NO: 65 - DNA, 23 bp, Spodoptera frugiperda
aagtgtcctgggcttgagttcca

SEQ ID NO: 66 - RNA, 19 nt, Spodoptera frugiperda
guguccugggcuugaguuc

SEQ ID NO: 67 - RNA, 19 nt, Spodoptera frugiperda
gaacucaagcccaggacac

SEQ ID NO: 68 - DNA, 23 bp, Spodoptera frugiperda
aagaagaagctcctccacgtgtt

SEQ ID NO: 69 - RNA, 19 nt, Spodoptera frugiperda
gaagaagcuccuccacgug

SEQ ID NO: 70 - RNA, 19 nt, Spodoptera frugiperda
cacguggaggagcuucuuc

SEQ ID NO: 71 - DNA, 23 bp, Spodoptera frugiperda
aaggtcgctgacgagaacaagga

SEQ ID NO: 72 - RNA, 19 nt, Spodoptera frugiperda
ggucgcugacgagaacaag

SEQ ID NO: 73 - RNA, 19 nt, Spodoptera frugiperda
cuuguucucgucagcgacc

SEQ ID NO: 74 - DNA, 23 bp, Spodoptera frugiperda
aatgtcctggggctgagtttcaa

SEQ ID NO: 75 - RNA, 19 nt, Spodoptera frugiperda
uguccuggggcugaguuuc

SEQ ID NO: 76 - RNA, 19 nt, Spodoptera frugiperda
gaaacucagccccaggaca

SEQ ID NO: 77 - DNA, 23 bp, Spodoptera frugiperda
aagaataagctcctccacgtgtt

SEQ ID NO: 78 - RNA, 19 nt, Spodoptera frugiperda
gaauaagcuccuccacgug

SEQ ID NO: 79 - RNA, 19 nt, Spodoptera frugiperda
cacguggaggagcuuauuc

SEQ ID NO: 80 - DNA, 23 bp, Spodoptera frugiperda
aatttgtcgaggagaccctattg

SEQ ID NO: 81 - RNA, 19 nt, Spodoptera frugiperda
uuugucgaggagacccuau

SEQ ID NO: 82 - RNA, 19 nt, Spodoptera frugiperda
auagggucuccucgacaaa

SEQ ID NO: 83 - DNA, 23 bp, Spodoptera frugiperda
aagttcgcgttcactcttgaaga

SEQ ID NO: 84 - RNA, 19 nt, Spodoptera frugiperda
guucgcguucacucuugaa

SEQ ID NO: 85 - RNA, 19 nt, Spodoptera frugiperda
uucaagagugaacgcgaac

SEQ ID NO: 86 - DNA, 23 bp, Spodoptera frugiperda
aactgccccttaacctcatctat

SEQ ID NO: 87 - RNA, 19 nt, Spodoptera frugiperda
cugccccuuaaccucaucu

SEQ ID NO: 88 - RNA, 19 nt, Spodoptera frugiperda
agaugagguuaaggggcag

SEQ ID NO: 89 - DNA, 23 bp, Spodoptera frugiperda
aatcacgctgaaaccactgtata

SEQ ID NO: 90 - RNA, 19 nt, Spodoptera frugiperda
ucacgcugaaaccacugua

SEQ ID NO: 91 - RNA, 19 nt, Spodoptera frugiperda
uacagugguuucagcguga

SEQ ID NO: 92 - DNA, 23 bp, Spodoptera frugiperda
aaaatatggcgcgcctattgttt

SEQ ID NO: 93 - RNA, 19 nt, Spodoptera frugiperda
aauauggcgcgccuauugu

SEQ ID NO: 94 - RNA, 19 nt, Spodoptera frugiperda
acaauaggcgcgccauauu

SEQ ID NO: 95 - DNA, 23 bp, Spodoptera frugiperda
aacgttctcggtctttcactgct

SEQ ID NO: 96 - RNA, 19 nt, Spodoptera frugiperda
cguucucggucuuucacug

SEQ ID NO: 97 - RNA, 19 nt, Spodoptera frugiperda
cagugaaagaccgagaacg

SEQ ID NO: 98 - DNA, 23 bp, Spodoptera frugiperda
aagtcatcgttccaagtctacct

SEQ ID NO: 99 - RNA, 19 nt, Spodoptera frugiperda
gucaucguuccaagucuac

SEQ ID NO: 100 - RNA, 19 nt, Spodoptera frugiperda
guagacuuggaacgaugac

SEQ ID NO: 101 - DNA, 23 bp, Spodoptera frugiperda
aaccccttgaatgttaaggtcgg

SEQ ID NO: 102 - RNA, 19 nt, Spodoptera frugiperda
ccccuugaauguuaagguc

SEQ ID NO: 103 - RNA, 19 nt, Spodoptera frugiperda
gaccuuaacauucaagggg

SEQ ID NO: 104 - DNA, 23 bp, Spodoptera frugiperda
aagtacaccatgttgcaagtatg

SEQ ID NO: 105 - RNA, 19 nt, Spodoptera frugiperda
guacaccauguugcaagua

SEQ ID NO: 106 - RNA, 19 nt, Spodoptera frugiperda
uacuugcaacaugguguac

SEQ ID NO: 107 - DNA, 23 bp, Spodoptera frugiperda
aacgtgtccatgatggctgactc

SEQ ID NO: 108 - RNA, 19 nt, Spodoptera frugiperda
cguguccaugauggcugac

SEQ ID NO: 109 - RNA, 19 nt, Spodoptera frugiperda
gucagccaucauggacacg

SEQ ID NO: 110 - DNA, 23 bp, Spodoptera frugiperda
aaacctacaaaatggccgaaaac

SEQ ID NO: 111 - RNA, 19 nt, Spodoptera frugiperda
accuacaaaauggccgaaa

SEQ ID NO: 112 - RNA, 19 nt, Spodoptera frugiperda
uuucggccauuuuguaggu

SEQ ID NO: 113 - DNA, 23 bp, Spodoptera frugiperda
aatctacggacccttctttggag

SEQ ID NO: 114 - RNA, 19 nt, Spodoptera frugiperda
ucuacggacccuucuuugg

SEQ ID NO: 115 - RNA, 19 nt, Spodoptera frugiperda
ccaaagaaggguccguaga

SEQ ID NO: 116 - DNA, 23 bp, Spodoptera frugiperda
aactctgacgtcatcatctacgt

SEQ ID NO: 117 - RNA, 19 nt, Spodoptera frugiperda
cucugacgucaucaucuac

SEQ ID NO: 118 - RNA, 19 nt, Spodoptera frugiperda
guagaugaugacgucagag

SEQ ID NO: 119 - DNA, 23 bp, Spodoptera frugiperda
aagtgcttgggtaaccccgacag

SEQ ID NO: 120 - RNA, 19 nt, Spodoptera frugiperda
gugcuuggguaaccccgac

SEQ ID NO: 121 - RNA, 19 nt, Spodoptera frugiperda
gucgggguuacccaagcac

SEQ ID NO: 122 - DNA, 23 bp, Spodoptera frugiperda
aactggctcatctcctacagcaa

SEQ ID NO: 123 - RNA, 19 nt, Spodoptera frugiperda
cuggcucaucuccuacagc

SEQ ID NO: 124 - RNA, 19 nt, Spodoptera frugiperda
gcuguaggagaugagccag

SEQ ID NO: 125 - DNA, 23 bp, Spodoptera frugiperda
aaacagtgcgtcgtaatatattc

SEQ ID NO: 126 - RNA, 19 nt, Spodoptera frugiperda
acagugcgucguaauauau

SEQ ID NO: 127 - RNA, 19 nt, Spodoptera frugiperda
auauauuacgacgcacugu

SEQ ID NO: 128 - DNA, 23 bp, Spodoptera frugiperda
aaggcacatggtccttcactgat

SEQ ID NO: 129 - RNA, 19 nt, Spodoptera frugiperda
ggcacaugguccuucacug

SEQ ID NO: 130 - RNA, 19 nt, Spodoptera frugiperda
cagugaaggaccaugugcc

SEQ ID NO: 131 - DNA, 23 bp, Spodoptera frugiperda
aacaccatgaccctcgtgtacaa

SEQ ID NO: 132 - RNA, 19 nt, Spodoptera frugiperda
caccaugacccucguguac

SEQ ID NO: 133 - RNA, 19 nt, Spodoptera frugiperda
guacacgagggucauggug

SEQ ID NO: 134 - DNA, 23 bp, Spodoptera frugiperda
aacgaggccggatctcttaagca

SEQ ID NO: 135 - RNA, 19 nt, Spodoptera frugiperda
cgaggccggaucucuuaag

SEQ ID NO: 136 - RNA, 19 nt, Spodoptera frugiperda
cuuaagagauccggccucg

SEQ ID NO: 137 - DNA, 23 bp, Spodoptera frugiperda
aacttcacacataactagacaaa

SEQ ID NO: 138 - RNA, 19 nt, Spodoptera frugiperda
cuucacacauaacuagaca

SEQ ID NO: 139 - RNA, 19 nt, Spodoptera frugiperda
ugucuaguuaugugugaag

SEQ ID NO: 140 - DNA, 23 bp, Spodoptera frugiperda
aatgcgtggcgatttcaaactta

SEQ ID NO: 141 - RNA, 19 nt, Spodoptera frugiperda
uuagaaauuauaagcccag

SEQ ID NO: 142 - RNA, 19 nt, Spodoptera frugiperda
cugggcuuauaauuucuaa

SEQ ID NO: 143 - DNA, 23 bp, Spodoptera frugiperda
aaaaaacacagaccacgttcaca

SEQ ID NO: 144 - RNA, 19 nt, Spodoptera frugiperda
aaaacacagaccacguuca

SEQ ID NO: 145 - RNA, 19 nt, Spodoptera frugiperda
ugaacguggucuguguuuu

SEQ ID NO: 146 - DNA, 23 bp, Spodoptera frugiperda
aatcgatggtggtgttattcgct

SEQ ID NO: 147 - RNA, 19 nt, Spodoptera frugiperda
ucgauggugguguuauucg

SEQ ID NO: 148 - RNA, 19 nt, Spodoptera frugiperda
cgaauaacaccaccaucga

SEQ ID NO: 149 - DNA, 23 bp, Spodoptera frugiperda
aaagaaaatgctacgcgttacga

SEQ ID NO: 150 - RNA, 19 nt, Spodoptera frugiperda
agaaaaugcuacgcguuac

SEQ ID NO: 151 - RNA, 19 nt, Spodoptera frugiperda
guaacgcguagcauuuucu

SEQ ID NO: 152 - DNA, 23 bp, Spodoptera frugiperda
aacccttggacactactggaaga

SEQ ID NO: 153 - RNA, 19 nt, Spodoptera frugiperda
cccuuggacacuacuggaa

SEQ ID NO: 154 - RNA, 19 nt, Spodoptera frugiperda
uuccaguaguguccaaggg

SEQ ID NO: 155 - DNA, 23 bp, Spodoptera frugiperda
aaggatcctatgtgtaccaggtt

SEQ ID NO: 156 - RNA, 19 nt, Spodoptera frugiperda
ggauccuauguguaccagg

SEQ ID NO: 157 - RNA, 19 nt, Spodoptera frugiperda
ccugguacacauaggaucc

SEQ ID NO: 158 - DNA, 23 bp, Spodoptera frugiperda
aaactcggcacacaacacaatgg

SEQ ID NO: 159 - RNA, 19 nt, Spodoptera frugiperda
acucggcacacaacacaau

SEQ ID NO: 160 - RNA, 19 nt, Spodoptera frugiperda
auuguguugugugccgagu

SEQ ID NO: 161 - DNA, 23 bp, Spodoptera frugiperda
aatacgaagatatctgcccttcc

SEQ ID NO: 162 - RNA, 19 nt, Spodoptera frugiperda
uacgaagauaucugcccuu

SEQ ID NO: 163 - RNA, 19 nt, Spodoptera frugiperda
aagggcagauaucuucgua

SEQ ID NO: 164 - DNA, 23 bp, Spodoptera frugiperda
aatcaacagctcttacataaatg

SEQ ID NO: 165 - RNA, 19 nt, Spodoptera frugiperda
ucaacagcucuuacauaaa

SEQ ID NO: 166 - RNA, 19 nt, Spodoptera frugiperda
uuuauguaagagcuguuga

SEQ ID NO: 167 - DNA, 23 bp, Spodoptera frugiperda
aaagaagatcagaagattggccg

SEQ ID NO: 168 - RNA, 19 nt, Spodoptera frugiperda
agaagaucagaagauuggc

SEQ ID NO: 169 - RNA, 19 nt, Spodoptera frugiperda
gccaaucuucugaucuucu

SEQ ID NO: 170 - DNA, 23 bp, Spodoptera frugiperda
aaaagccgtctgctatccaacaa

SEQ ID NO: 171 - RNA, 19 nt, Spodoptera frugiperda
aagccgucugcuauccaac

SEQ ID NO: 172 - RNA, 19 nt, Spodoptera frugiperda
guuggauagcagacggcuu

SEQ ID NO: 173 - DNA, 23 bp, Spodoptera frugiperda
aatgctaaatgccatgcttgcat

SEQ ID NO: 174 - RNA, 19 nt, Spodoptera frugiperda
ugcuaaaugccaugcuugc

SEQ ID NO: 175 - RNA, 19 nt, Spodoptera frugiperda
gcaagcauggcauuuagca

SEQ ID NO: 176 - DNA, 23 bp, Spodoptera frugiperda
aagatcagaagattggccggaag

SEQ ID NO: 177 - RNA, 19 nt, Spodoptera frugiperda
gaucagaagauuggccgga

SEQ ID NO: 178 - RNA, 19 nt, Spodoptera frugiperda
uccggccaaucuucugauc

SEQ ID NO: 179 - DNA, 23 bp, Spodoptera frugiperda
aattcttcagcaaatcgatacca

SEQ ID NO: 180 - RNA, 19 nt, Spodoptera frugiperda
uucuucagcaaaucgauac

SEQ ID NO: 181 - RNA, 19 nt, Spodoptera frugiperda
guaucgauuugcugaagaa

SEQ ID NO: 182 - DNA, 23 bp, Spodoptera frugiperda
aaatgctgtcaagaggatttaaa

SEQ ID NO: 183 - RNA, 19 nt, Spodoptera frugiperda
augcugucaagaggauuua

SEQ ID NO: 184 - RNA, 19 nt, Spodoptera frugiperda
uaaauccucuugacagcau

SEQ ID NO: 185 - DNA, 23 bp, Spodoptera frugiperda
aagctcgagacttgctcttgatg

SEQ ID NO: 186 - RNA, 19 nt, Spodoptera frugiperda
gcucgagacuugcucuuga

SEQ ID NO: 187 - RNA, 19 nt, Spodoptera frugiperda
ucaagagcaagucucgagc

SEQ ID NO: 188 - DNA, 23 bp, Spodoptera frugiperda
aactgttagctcaaggtctgcta

SEQ ID NO: 189 - RNA, 19 nt, Spodoptera frugiperda
cuguuagcucaaggucugc

SEQ ID NO: 190 - RNA, 19 nt, Spodoptera frugiperda
gcagaccuugagcuaacag

SEQ ID NO: 191 - DNA, 23 bp, Spodoptera frugiperda
aagactttctatcagaatttgcg

SEQ ID NO: 192 - RNA, 19 nt, Spodoptera frugiperda
gacuuucuaucagaauuug

SEQ ID NO: 193 - RNA, 19 nt, Spodoptera frugiperda
caaauucugauagaaaguc

SEQ ID NO: 194 - DNA, 23 bp, Spodoptera frugiperda
aaacttaatcatggacgacgaca

SEQ ID NO: 195 - RNA, 19 nt, Spodoptera frugiperda
acuuaaucauggacgacga

SEQ ID NO: 196 - RNA, 19 nt, Spodoptera frugiperda
ucgucguccaugauuaagu

SEQ ID NO: 197 - DNA, 23 bp, Spodoptera frugiperda
aaagaagaagaagaagaagggag

SEQ ID NO: 198 - RNA, 19 nt, Spodoptera frugiperda
agaagaagaagaagaaggg

SEQ ID NO: 199 - RNA, 19 nt, Spodoptera frugiperda
cccuucuucuucuucuucu

SEQ ID NO: 200 - DNA, 23 bp, Spodoptera frugiperda
aagatcaagagaatgtcgaggat

SEQ ID NO: 201 - RNA, 19 nt, Spodoptera frugiperda
gaucaagagaaugucgagg

SEQ ID NO: 202 - RNA, 19 nt, Spodoptera frugiperda
ccucgacauucucuugauc

SEQ ID NO: 203 - DNA, 23 bp, Spodoptera frugiperda
aaaatcgtcggttttagcgacgt

SEQ ID NO: 204 - RNA, 19 nt, Spodoptera frugiperda
aaucgucgguuuuagcgac

SEQ ID NO: 205 - RNA, 19 nt, Spodoptera frugiperda
gucgcuaaaaccgacgauu

SEQ ID NO: 206 - DNA, 23 bp, Spodoptera frugiperda
aactgtcaataggcagtatgcgt

SEQ ID NO: 207 - RNA, 19 nt, Spodoptera frugiperda
cugucaauaggcaguaugc

SEQ ID NO: 208 - RNA, 19 nt, Spodoptera frugiperda
gcauacugccuauugacag

SEQ ID NO: 209 - DNA, 23 bp, Spodoptera frugiperda
aacctgtaccaacagaccactgg

SEQ ID NO: 210 - RNA, 19 nt, Spodoptera frugiperda
ccuguaccaacagaccacu

SEQ ID NO: 211 - RNA, 19 nt, Spodoptera frugiperda
aguggucuguugguacagg

SEQ ID NO: 212 - DNA, 23 bp, Spodoptera frugiperda
aaccaaaaatgggcaaggaaaag

SEQ ID NO: 213 - RNA, 19 nt, Spodoptera frugiperda
ccaaaaaugggcaaggaaa

SEQ ID NO: 214 - RNA, 19 nt, Spodoptera frugiperda
uuuccuugcccauuuuugg

SEQ ID NO: 215 - DNA, 23 bp, Spodoptera frugiperda
aacgtggtatcaccatcgatatt

SEQ ID NO: 216 - RNA, 19 nt, Spodoptera frugiperda
cgugguaucaccaucgaua

SEQ ID NO: 217 - RNA, 18 nt, Spodoptera frugiperda
uaucgauggugauaccac

SEQ ID NO: 218 - DNA, 23 bp, Spodoptera frugiperda
aacaaaatggactccactgagcc

SEQ ID NO: 219 - RNA, 19 nt, Spodoptera frugiperda
caaaauggacuccacugag

SEQ ID NO: 220 - RNA, 19 nt, Spodoptera frugiperda
cucaguggaguccauuuug

SEQ ID NO: 221 - DNA, 23 bp, Spodoptera frugiperda
aatccgtgactaaccaaaaatgg

SEQ ID NO: 222 - RNA, 19 nt, Spodoptera frugiperda
uccgugacuaaccaaaaau

SEQ ID NO: 223 - RNA, 19 nt, Spodoptera frugiperda
auuuuugguuagucacgga

SEQ ID NO: 224 - DNA, 23 bp, Spodoptera frugiperda
aacattgtcgtcattggacacgt

SEQ ID NO: 225 - RNA, 19 nt, Spodoptera frugiperda
cauugucgucauuggacac

SEQ ID NO: 226 - RNA, 19 nt, Spodoptera frugiperda
guguccaaugacgacaaug

SEQ ID NO: 227 - DNA, 23 bp, Spodoptera frugiperda
aatttgtgagactggtggccgaa

SEQ ID NO: 228 - RNA, 19 nt, Spodoptera frugiperda
uuugugagacugguggccg

SEQ ID NO: 229 - RNA, 19 nt, Spodoptera frugiperda
cggccaccagucucacaaa

SEQ ID NO: 230 - DNA, 23 bp, Spodoptera frugiperda
aatctgattgtattcgccccctc

SEQ ID NO: 231 - RNA, 19 nt, Spodoptera frugiperda
ucugauuguauucgccccc

SEQ ID NO: 232 - RNA, 19 nt, Spodoptera frugiperda
gggggcgaauacaaucaga

SEQ ID NO: 233 - DNA, 23 bp, Spodoptera frugiperda
aacactctagttctgcctattct

SEQ ID NO: 234 - RNA, 19 nt, Spodoptera frugiperda
cacucuaguucugccuauu

SEQ ID NO: 235 - RNA, 19 nt, Spodoptera frugiperda
aauaggcagaacuagagug

SEQ ID NO: 236 - DNA, 23 bp, Spodoptera frugiperda
aacacacatcacaatggcggata

SEQ ID NO: 237 - RNA, 19 nt, Spodoptera frugiperda
cacacaucacaauggcgga

SEQ ID NO: 238 - RNA, 19 nt, Spodoptera frugiperda
uccgccauugugaugugug

SEQ ID NO: 239 - DNA, 23 bp, Spodoptera frugiperda
aaggatggcatcatcggcaagaa

SEQ ID NO: 240 - RNA, 19 nt, Spodoptera frugiperda
ggauggcaucaucggcaag

SEQ ID NO: 241 - RNA, 19 nt, Spodoptera frugiperda
cuugccgaugaugccaucc

SEQ ID NO: 242 - DNA, 23 bp, Spodoptera frugiperda
aaaggcttcatcgacaccgcgaa

SEQ ID NO: 243 - RNA, 19 nt, Spodoptera frugiperda
aggcuucaucgacaccgcg

SEQ ID NO: 244 - RNA, 19 nt, Spodoptera frugiperda
cgcggugucgaugaagccu

SEQ ID NO: 245 - DNA, 23 bp, Spodoptera frugiperda
aaactccaattataacctactag

SEQ ID NO: 246 - RNA, 19 nt, Spodoptera frugiperda
acuccaauuauaaccuacu

SEQ ID NO: 247 - RNA, 19 nt, Spodoptera frugiperda
aguagguuauaauuggagu

SEQ ID NO: 248 - DNA, 23 bp, Spodoptera frugiperda
aagtacaaggatctgatcggcaa

SEQ ID NO: 249 - RNA, 19 nt, Spodoptera frugiperda
guacaaggaucugaucggc

SEQ ID NO: 250 - RNA, 19 nt, Spodoptera frugiperda
gccgaucagauccuuguac

SEQ ID NO: 251 - DNA, 23 bp, Spodoptera frugiperda
aagactttcttcatgtggcccat

SEQ ID NO: 252 - RNA, 19 nt, Spodoptera frugiperda
gacuuucuucauguggccc

SEQ ID NO: 253 - RNA, 19 nt, Spodoptera frugiperda
gggccacaugaagaaaguc

SEQ ID NO: 254 - DNA, 23 bp, Spodoptera frugiperda
aaacaaagtatcgcctacaccgc

SEQ ID NO: 255 - RNA, 19 nt, Spodoptera frugiperda
acaaaguaucgccuacacc

SEQ ID NO: 256 - RNA, 19 nt, Spodoptera frugiperda
gguguaggcgauacuuugu

SEQ ID NO: 257 - DNA, 23 bp, Spodoptera frugiperda
aatagcgtcgatcttcaacgact

SEQ ID NO: 258 - RNA, 19 nt, Spodoptera frugiperda
uagcgucgaucuucaacga

SEQ ID NO: 259 - RNA, 19 nt, Spodoptera frugiperda
ucguugaagaucgacgcua

SEQ ID NO: 260 - DNA, 23 bp, Spodoptera frugiperda
aactcatagagcttgatgtgtgg

SEQ ID NO: 261 - RNA, 19 nt, Spodoptera frugiperda
cucauagagcuugaugugu

SEQ ID NO: 262 - RNA, 19 nt, Spodoptera frugiperda
acacaucaagcucuaugag

SEQ ID NO: 263 - DNA, 23 bp, Spodoptera frugiperda
aagatgtggatgacgtcactggt

SEQ ID NO: 264 - RNA, 19 nt, Spodoptera frugiperda
gauguggaugacgucacug

SEQ ID NO: 265 - RNA, 19 nt, Spodoptera frugiperda
cagugacgucauccacauc

SEQ ID NO: 266 - DNA, 23 bp, Spodoptera frugiperda
aaccttcctgattctcttctgtg

SEQ ID NO: 267 - RNA, 19 nt, Spodoptera frugiperda
ccuuccugauucucuucug

SEQ ID NO: 268 - RNA, 19 nt, Spodoptera frugiperda
cagaagagaaucaggaagg

SEQ ID NO: 269 - DNA, 23 bp, Spodoptera frugiperda
aacagtgcttgtgataagtgaac

SEQ ID NO: 270 - RNA, 19 nt, Spodoptera frugiperda
cagugcuugugauaaguga

SEQ ID NO: 271 - RNA, 19 nt, Spodoptera frugiperda
ucacuuaucacaagcacug

SEQ ID NO: 272 - DNA, 23 bp, Spodoptera frugiperda
aagttaatggtgactgccctcga

SEQ ID NO: 273 - RNA, 19 nt, Spodoptera frugiperda
guuaauggugacugcccuc

SEQ ID NO: 274 - RNA, 19 nt, Spodoptera frugiperda
gagggcagucaccauuaac

SEQ ID NO: 275 - DNA, 23 bp, Spodoptera frugiperda
aataaagcgatgaccccatagga

SEQ ID NO: 276 - RNA, 19 nt, Spodoptera frugiperda
uaaagcgaugaccccauag

SEQ ID NO: 277 - RNA, 19 nt, Spodoptera frugiperda
cuauggggucaucgcuuua

SEQ ID NO: 278 - DNA, 23 bp, Spodoptera frugiperda
aaacggtactgcagcaaaaagac

SEQ ID NO: 279 - RNA, 19 nt, Spodoptera frugiperda
acgguacugcagcaaaaag

SEQ ID NO: 280 - RNA, 19 nt, Spodoptera frugiperda
cuuuuugcugcaguaccgu

SEQ ID NO: 281 - DNA, 23 bp, Spodoptera frugiperda
aagctgcatacttcttggctctc

SEQ ID NO: 282 - RNA, 19 nt, Spodoptera frugiperda
gcugcauacuucuuggcuc

SEQ ID NO: 283 - RNA, 19 nt, Spodoptera frugiperda
gagccaagaaguaugcagc

SEQ ID NO: 284 - DNA, 23 bp, Spodoptera frugiperda
aaatgtttacagagacgcgatga

SEQ ID NO: 285 - RNA, 19 nt, Spodoptera frugiperda
auguuuacagagacgcgau

SEQ ID NO: 286 - RNA, 19 nt, Spodoptera frugiperda
aucgcgucucuguaaacau

SEQ ID NO: 287 - DNA, 23 bp, Spodoptera frugiperda
aacgtcgatcttaccgagttcca

SEQ ID NO: 288 - RNA, 19 nt, Spodoptera frugiperda
cgucgaucuuaccgaguuc

SEQ ID NO: 289 - RNA, 19 nt, Spodoptera frugiperda
gaacucgguaagaucgacg

SEQ ID NO: 290 - DNA, 23 bp, Spodoptera frugiperda
aattcaaaatgcgtgagtgcatc

SEQ ID NO: 291 - RNA, 19 nt, Spodoptera frugiperda
uucaaaaugcgugagugca

SEQ ID NO: 292 - RNA, 19 nt, Spodoptera frugiperda
ugcacucacgcauuuugaa

SEQ ID NO: 293 - DNA, 23 bp, Spodoptera frugiperda
aaatcgtagacctagtcctcgac

SEQ ID NO: 294 - RNA, 19 nt, Spodoptera frugiperda
aucguagaccuaguccucg

SEQ ID NO: 295 - RNA, 19 nt, Spodoptera frugiperda
cgaggacuaggucuacgau

SEQ ID NO: 296 - DNA, 23 bp, Spodoptera frugiperda
aaactcaattcaaaatgcgtgag

SEQ ID NO: 297 - RNA, 19 nt, Spodoptera frugiperda
acucaauucaaaaugcgug

SEQ ID NO: 298 - RNA, 19 nt, Spodoptera frugiperda
cacgcauuuugaauugagu

SEQ ID NO: 299 - DNA, 23 bp, Spodoptera frugiperda
aacttatcactggtaaggaagat

SEQ ID NO: 300 - RNA, 18 nt, Spodoptera frugiperda
cuuaucacugguaaggaa

SEQ ID NO: 301 - RNA, 19 nt, Spodoptera frugiperda
cuuccuuaccagugauaag

SEQ ID NO: 302 - DNA, 23 bp, Spodoptera frugiperda
aagagttacgaaccgtcaccata

SEQ ID NO: 303 - RNA, 19 nt, Spodoptera frugiperda
gaguuacgaaccgucacca

SEQ ID NO: 304 - RNA, 19 nt, Spodoptera frugiperda
uggugacgguucguaacuc

SEQ ID NO: 305 - DNA, 23 bp, Spodoptera frugiperda
aaacttagtccggataatgaacc

SEQ ID NO: 306 - RNA, 19 nt, Spodoptera frugiperda
acuuaguccggauaaugaa

SEQ ID NO: 307 - RNA, 19 nt, Spodoptera frugiperda
uucauuauccggacuaagu

SEQ ID NO: 308 - DNA, 23 bp, Spodoptera frugiperda
aaggcgatgtacgagaacctgtt

SEQ ID NO: 309 - RNA, 19 nt, Spodoptera frugiperda
ggcgauguacgagaaccug

SEQ ID NO: 310 - RNA, 19 nt, Spodoptera frugiperda
cagguucucguacaucgcc

SEQ ID NO: 311 - DNA, 23 bp, Spodoptera frugiperda
aacgacaagatgctgaaggagac

SEQ ID NO: 312 - RNA, 19 nt, Spodoptera frugiperda
cgacaagaugcugaaggag

SEQ ID NO: 313 - RNA, 19 nt, Spodoptera frugiperda
cuccuucagcaucuugucg

SEQ ID NO: 314 - DNA, 23 bp, Spodoptera frugiperda
aagataaaggtcgcgtgtggacc

SEQ ID NO: 315 - RNA, 19 nt, Spodoptera frugiperda
gauaaaggucgcgugugga

SEQ ID NO: 316 - RNA, 19 nt, Spodoptera frugiperda
uccacacgcgaccuuuauc

SEQ ID NO: 317 - DNA, 23 bp, Spodoptera frugiperda
aatgtcaagactgatccaaacac

SEQ ID NO: 318 - RNA, 19 nt, Spodoptera frugiperda
ugucaagacugauccaaac

SEQ ID NO: 319 - RNA, 19 nt, Spodoptera frugiperda
guuuggaucagucuugaca

SEQ ID NO: 320 - DNA, 23 bp, Spodoptera frugiperda
aacattcgagtctgaacaggtgg

SEQ ID NO: 321 - RNA, 19 nt, Spodoptera frugiperda
cauucgagucugaacaggu

SEQ ID NO: 322 - RNA, 19 nt, Spodoptera frugiperda
accuguucagacucgaaug